(19) United States
(12) Patent Application Publication
(10) Pub. No.: US 2006/0106442 A1
(43) Pub. Date: May 18, 2006

(54) Devices and methods for treating cardiac pathologies

(75) Inventors: Kelly Richardson, Menlo Park, CA (US); Amrish Walke, Santa Clara, CA (US); Russell Woo, Palo Alto, CA (US); Daniel Francis, Mountain View, CA (US); Evan Anderson, Sunnyvale, CA (US); Jeremy Johnson, Santa Rosa, CA (US)

Correspondence Address: BELL & ASSOCIATES, 416 FUNSTON ST., SUITE 100, SAN FRANCISCO, CA, 94118, US

(73) Assignee: The Board of Trustees of the Leland Stanford Junior University

(21) Appl. No.: 11/133,581

(22) Filed: May 19, 2005

Related U.S. Application Data
(60) Provisional application No. 60/572,715, filed on May 19, 2004.

(51) Int. Cl. A61N 1/05

(52) U.S. Cl. 607/119

(57) ABSTRACT

The invention relates to heart treatment devices and methods for performing diagnostic or therapeutic procedures in the region between a bodily organ and a covering of the bodily organ. The invention can be used for example to perform a diagnostic or therapeutic procedure in the pericardial space.

The present application claims priority to U.S. Provisional Patent Application Ser. No. 60/572,715 entitled “Novel Methods And Devices For Cardiac Leads And Cardiac Lead Placement”, filed May 19, 2004, which is herein incorporated by reference in its entirety for all purposes.

### FIELD OF THE INVENTION

The present invention relates to devices and methods to prevent or treat an individual having congestive heart failure and related conditions.

The present invention relates to devices and methods for performing diagnostic or therapeutic procedures in the region between a bodily organ and a covering of the bodily organ. The present invention can be used for example to perform a diagnostic or therapeutic procedure in the pericardial space. More specifically, the present invention can be used for implanting one or more electrical leads on the cardiac myocardium.

### BACKGROUND

Several diagnostic or therapeutic procedures can be performed by accessing the region between an organ and an anatomical covering around the organ. Examples of such pairs of organ-covering are heart-pericardium, brain-dura mater, and the spinal cord-dura mater. One example of a therapeutic procedure that can be performed by accessing the pericardial region (region between heart and pericardium) is cardiac pacing for treating congestive heart failure (CHF).

A clinical method and procedure for treating CHF involves inducing pacing of the heart that generally results in improved outcomes by improving heart pump function. One primary anatomical approach used by cardiologists is through the coronary sinus. More recently, cardiologists have been experimenting with a sub-xiphoid approach. Both of these approaches have difficulties and substantial drawbacks.

Congestive heart failure (CHF) affects 4.8 million Americans and causes 266,000 deaths annually in the US. It can be the result of myocardial infarctions, longstanding hypertension, infection, alcohol abuse, genetic and in some cases, idiopathic (no clear source found). It has historically been treated with medications. Published data from the Framingham cohort indicate that, irrespective of age, men and women have an almost equal (20%) likelihood of developing CHF over a lifetime (Young (2004) Rev. Cardiovasc. Med. 5 (Suppl. 1): S3-S9; Lloyd-Jones et al. (2002) Circulation 106: 3068-3072). The 5-year mortality rate remains high, at 59%.

Recently, pacemaker technology has been used as a new therapy to treat CHF. When CHF occurs the heart dilates and pumps in a dysynchronous manner resulting in inefficient pumping of blood out of the heart to the rest of the body. With the dysynchronous beating much of the blood flows backwards filling the lungs with fluid and causing CHF symptoms such as shortness of breath, dyspnea, and swelling. By pacing both the left and right sides of the heart (biventricular pacing) the uncoordinated pumping of the heart is repaired. This therapy is called Cardiac Resynchronization Therapy (CRT). CRT or biventricular pacing is achieved by placing one lead through the coronary sinus to a coronary vein to pace the left ventricle (LV). The other lead is placed endocardially in the right ventricle (RV). Many patients with CHF and dysynchronous pumping have a widened QRS on the electrocardiogram called a bundle branch block. Initially, CRT was offered only to those with a widened QRS and CHF; however, it is now known that many patients with a normal QRS have evidence of dysynchrony on echocardiograms and most likely would also benefit from CRT.

Randomized multicentre studies show significant improvement of functional capacity, quality of life and left ventricular systolic function in patients with severe heart failure and bundle branch block. In one study, a significant reduction of 77% of hospitalisation days has been demonstrated. Biventricular pacing can be combined with intracardiac defibrillators (ie the ability to “shock” the heart) by adding defibrillator coils or electrodes on the right ventricular lead. Using combined implantable biventricular pacing and cardioverter defibrillator leads resulted in a 43% reduction of mortality in patients with severe heart failure compared to optimal pharmacological treatment only. (Faerestrand (2004) Tidsskr. Nor. Laegeforen. 124: 1111-1115.)

Biventricular pacing has been shown to improve heart failure outcomes in patients having Class III and Class IV heart failure and mechanical dysynchrony represented by a wide QRS wave on the electrocardiogram. Placement of the pacing leads is usually done using a minimally-invasive catheterization. Placement of the LV lead is frought with limitations and potentials for complications. One of the most time-consuming and difficult parts of the method of implantation of such a device is the step of placing the left ventricular pacing lead through the coronary sinus. This is due to a wide anatomical variation between patients' venous anatomy. Once in the coronary sinus placement of the lead is limited by the venous anatomy. Some patients have so few branches that placement of the lead is impossible. In addition, one theory for why some people do not respond to biventricular pacing is the inability to place the lead in the optimal location (i.e. the point of latest contraction on the LV determined by echocardiogram). Once in a branch, there is often diaphragmatic stimulation due to the close location of the phrenic nerve. When there is diaphragmatic stimulation, the lead must be repositioned unless programming can eliminate the problem. In 6% of the cases the lead dislodges from its site requiring another surgery. In some cases the lead is unable to be placed and requires minimally invasive surgery to be placed epicardially via a thoroscopic incision.

Once the lead is positioned in an optimal branch of the CS, pacing threshold is determined using volts and pulse width (for example a potential difference of 2.5 V and a pulse width of 0.5 ms). Ideally, the chosen location will require the smallest amount of energy to pace the left ventricle. Since positioning is determined by venous anatomy the ideal thresholds may not be achieved. When higher amounts of energy are required, battery lifetime is shorter. In addition, ideally to achieve the most coordinated pumping, the lead should be placed at the point on the left ventricle that contracts latest during normal sinus rhythm. Many times this location cannot be achieved due to the limitations of coronary branches.

There exists an unmet medical need for a safer, more effective, and more reliable method of delivering CRT for patients with a cardiac pathology.

### BRIEF DESCRIPTION OF THE INVENTION

The present invention provides a catheter system for positioning an intrapericardial electrical catheter against a site upon the surface of a mammalian heart, the mammalian heart having an inner surface and an outer surface defining a wall, the catheter system comprising a first tube, the first tube having a proximal end and a distal end, and defining a first lumen within the longitudinal axis of the first tube, an attaching means having a proximal end and a distal end, wherein the proximal end of the attaching means is affixed to the first tube at or near the distal end of the first tube and the distal end of the attaching means is adapted for attaching the first tube to the wall of the mammalian heart, a second tube, the second tube having a distal end adapted for puncturing the wall of the mammalian heart and having a proximal end, wherein the second tube is shaped and adapted for placement within the first lumen, and an intrapericardial electrical catheter having a distal end and a proximal end, defining a second lumen within the longitudinal axis of the intrapericardial electrical catheter, wherein the intrapericardial electrical catheter is adapted for insertion through the first lumen, and wherein the distal end of the intrapericardial electrical catheter is shaped and adapted for placement against a site upon the surface of the mammalian heart. In another embodiment the distal end of the intrapericardial electrical catheter is shaped and adapted for puncturing the wall of the mammalian heart.

The various methods and devices disclosed in this patent applications may be used for pacing one or more heart chambers (LV, RV, right atrium, left atrium, etc.), defibrillating or more heart chambers (LV, RV, right atrium, left atrium, etc.), ablating a region of the heart or the surrounding vasculature, injecting a medication or biological materials such as stem cells, diagnose a source of arrhythmia, introduce or withdraw fluids, introduce substances or devices that prevent pathological expansion or dilation of heart tissue, accessing the coronary vasculature to perform a diagnostic or therapeutic procedure, measure electrical properties of the heart, and the like.

In one embodiment the distal end of the second tube comprises a perforator, the perforator selected from the group consisting of a needle, a hook, a pin, a fastener, or the like.

In one embodiment the attaching means is selected from the group consisting of stylet, a hook, a clip, a staple, an adhesive, a coil, a barb, a serrated blade or knife, a threaded screw, and a vacuum device. In an additional embodiment, the catheter system further comprises a guide-wire, the guide-wire comprising a material selected from the group consisting of a nickel-titanium alloy (such as NITINOL), stainless steel, and titanium.

In a further embodiment, the intrapericardial electrical catheter further comprises a magnetic tip.

In another embodiment the distal end of the intrapericardial electrical catheter further comprises attaching means selected from the group consisting of clips, hook, staples, adhesives, and a vacuum device and the attaching means are adapted for securing the intrapericardial electrical catheter to the outer surface of a heart myocardium.

In one embodiment the first catheter further comprises at least one electrode, the electrode selected from the group consisting of a pacing electrode and a defibrillating electrode.

In another embodiment the first tube further comprising an elongate flexing element, the elongate flexing element comprising a material selected from the group consisting of stainless steel, nickel-titanium alloy, and polymeric materials.

In an alternative embodiment the first tube further comprises a right ventricular pacing (RV) lead. In a further embodiment, the first tube further comprises a gasket ring, wherein the gasket ring is affixed near the distal end of the first tube and wherein in the gasket ring has a diameter more than that of the first tube. In a preferred embodiment the gasket ring is affixed to the first tube at a distance from the distal end of the first tube of about 1 mm less than the thickness of the wall of the mammalian organ.

In yet another alternative embodiment the second tube further comprises a right ventricular pacing (RV) lead. In a further embodiment, the second tube further comprises a gasket ring, wherein the gasket ring is affixed near the distal end of the second tube and wherein in the gasket ring has a diameter more than that of the second tube. In a preferred embodiment the gasket ring is affixed to the second tube at a distance from the distal end of the second tube of about 1 mm less than the thickness of the wall of the mammalian organ.

In an alternative embodiment, the gasket ring is affixed to the first tube at a distance from the distal end of the first tube of about 1 mm more than the thickness of the wall of the mammalian organ.

In an alternative embodiment, the gasket ring is affixed to the second tube at a distance from the distal end of the second tube of about 1 mm more than the thickness of the wall of the mammalian organ.

In a preferred embodiment the intrapericardial electrical catheter is concentrically disposed within the first lumen and comprises at least one first pair of electrodes, and the first pair of electrodes are in electrical conductivity to a power source at or near the proximal end of the intrapericardial electrical catheter.

In another embodiment the intrapericardial electrical catheter further comprises a catheter selected from the group consisting of an ablation catheter, a cryogenic catheter, a drug-delivery catheter, a cell-delivery catheter, or the like.

In one embodiment the attaching means of the catheter system is an electrode. In another embodiment the catheter system comprises a pacing lead and a shocking electrode. In another embodiment the catheter system comprises at least one electrode. In another embodiment the catheter system comprises a plurality of electrodes. In another embodiment the catheter system comprises a bending device, mechanism, or the like. In another embodiment the distal end of the second tube adapted for perforating the wall of the myocardium is a needle. In another embodiment the distal end of the second tube adapted for perforating the wall of the myocardium is a wire with a sharp distal tip. In another embodiment the distal end of the second tube adapted for perforating the wall of the myocardium comprises a lumen. In another embodiment the catheter system further comprising a guidewire that can be introduced through lumen of perforating device. In another embodiment the guidewire is a pressure wire (pressure transducer) to measure pressure. In another embodiment the guidewire comprises a lubricious coating on the outer surface. In another embodiment the guidewire is steerable. In another embodiment the guidewire is preshaped. In another embodiment the guidewire comprises a removable stylet. In another embodiment the intrapericardial electrical catheter comprises a distal fixation device, mechanism, or the like. In another embodiment the intrapericardial electrical catheter comprises one or more electrodes. In another embodiment the intrapericardial electrical catheter comprises a lumen. In another embodiment the intrapericardial electrical catheter comprises a lumen, and one or more perforations on its outer surface that are in fluid communication with the lumen. In another embodiment the intrapericardial electrical catheter comprises a shocking electrode. In another embodiment the intrapericardial electrical catheter comprises a Doppler and/or ultrasound transducer upon its distal end.

In another embodiment, the catheter system comprises an intrapericardial electrical catheter wherein the intrapericardial electrical catheter is concentrically disposed within the first lumen and comprises at least one first pair of electrodes, and the first pair of electrodes are in electrical conductivity to a power source at or near the proximal end of the intrapericardial electrical catheter. In a still further embodiment the first catheter further comprises at least one second pair of electrodes, the second pair of electrodes being disposed at or near the distal end of the second catheter and are in electrical conductivity to a power source at or near the proximal end of the first tube and wherein the wall of the mammalian organ conducts a current when a potential difference is created between the first pair of electrodes and the second pair of electrodes using the power source. In one embodiment the potential difference is not more than about 2.5 V at 0.5 ms. In a more preferred embodiment the potential difference is not more than about 2.0 V at 0.5 ms. In a most preferred embodiment the potential difference is not more than about 1.0 V at 0.5 ms.

In yet another embodiment the distal end of the first intrapericardial electrical catheter further comprises a marker, the marker selected from the group consisting of a dye, a radio-opaque material, a magnet, echogenic material, and an ion source.

In an alternative embodiment, the invention provides a catheter system for positioning an intrapericardial electrical catheter against a site upon the surface of a mammalian heart, the mammalian heart having an inner surface and an outer surface defining a wall, the catheter system comprising a first tube, the first tube having a proximal end and a distal end, and defining a first lumen within the longitudinal axis of the first tube, and an intrapericardial electrical catheter having a distal end and a proximal end, defining a second lumen within the longitudinal axis of the intrapericardial electrical catheter, wherein the intrapericardial electrical catheter is adapted for insertion through the first lumen, and wherein the distal end of the intrapericardial electrical catheter is shaped and adapted for placement against a site upon the surface of the mammalian organ. In one embodiment the catheter system further comprises an attaching means having a proximal end and a distal end, wherein the proximal end of the attaching means is affixed to the intrapericardial electrical catheter at or near the distal end of the intrapericardial electrical catheter and the distal end of the attaching means is adapted for attaching the intrapericardial electrical catheter to the wall of the mammalian heart,

In a further embodiment the catheter system comprises a second tube, the second tube having a distal end adapted to puncture the wall of the mammalian organ and having a proximal end, wherein the second tube is adapted for placement within the first lumen,

In a still further embodiment the proximal end and the distal end of the second tube defining a third lumen. In a yet further embodiment, the first tube further comprises an elongate flexing element comprising a material selected from the group consisting of stainless steel, nickel-titanium alloy, and polymeric materials.

In an alternative embodiment the invention provides a system for diagnosing, preventing, or treating a cardiac pathology by perforating through a cardiac tissue and placing a medical device or fluid in a heart chamber comprising a) an elongated, tubular delivery device having a delivery device lumen extending from a delivery device proximal region to a delivery device distal region and a distal fixation mechanism at the delivery device distal region adapted to be advanced into the heart and fixed to a selected site on the heart wall; b) an elongated perforating device having a perforating device distal tip adapted to be advanced through the delivery device lumen and perforate through the cardiac tissue into the heart chamber to form a perforation; and c) an elongate medical device for diagnosing, preventing or treating a cardiac pathology to be advanced through the perforation into the heart chamber. In a preferred embodiment, the cardiac pathology is an electrophysiological pathology.

In a still alternative embodiment the invention provides a system for diagnosing, preventing, or treating a cardiac pathology by perforating through a thoracic vascular tissue and placing a medical device or fluid in a heart chamber comprising: a) an elongated, tubular delivery device having a delivery device lumen extending from a delivery device proximal region to a delivery device distal region; b) an elongated perforating device having an perforating device distal tip adapted to be advanced through the delivery device lumen and perforate through the thoracic vascular tissue into the heart chamber to form a perforation; and c) an elongate medical device for diagnosing, preventing or treating a cardiac pathology to be advanced through the perforation into the heart chamber. In a preferred embodiment the cardiac pathology is an electrophysiological pathology.

In a yet still alternative embodiment the invention provides a system for delivering electrical energy to a location on the heart wall by perforating through a cardiac tissue and placing a medical device in the pericardial space comprising: a) an elongated, tubular delivery device having a delivery device lumen extending from a delivery device proximal region to a delivery device distal region and a distal fixation mechanism at the delivery device distal region adapted to be advanced into the heart and fixed to a selected site on the heart wall; b) an elongated perforating device having an perforating device distal tip adapted to be advanced through the delivery device lumen and perforate through the cardiac tissue into the pericardial space to form a perforation; and c) an elongate medical device for delivering electrical energy to be advanced through the perforation into the pericardial space.

In another alternative embodiment the invention provides a system for delivering electrical energy to a location on the heart wall by perforating through a thoracic vascular tissue and placing a medical device in the pericardial space comprising: a) an elongated, tubular delivery device having a delivery device lumen extending from a delivery device proximal region to a delivery device distal region; b) an elongated perforating device having an perforating device distal tip adapted to be advanced through the delivery device lumen and perforate through the thoracic vascular tissue into the pericardial space to form a perforation; and c) an elongate medical device for delivering electrical energy to be advanced through the perforation into the pericardial space.

In a still alternative embodiment the invention provides a system for delivering electrical energy to multiple cardiac locations by perforating through a cardiac tissue and placing a medical device comprising: a) an elongated, tubular delivery device having a delivery device lumen extending from a delivery device proximal region to a delivery device distal region; b) an elongated perforating device having an perforating device distal tip adapted to be advanced through the delivery device lumen and perforate through the cardiac tissue into the pericardial space to form a perforation; and c) an elongate medical device for delivering electrical energy to multiple cardiac locations to be advanced through the perforation into the pericardial space, wherein the elongate medical device comprises two or more distal electrodes for delivering electrical energy and two or more proximal electrodes for delivering electrical energy.

The invention also provides a method of using a catheter system to treat an individual having a cardiac pathology and that results in the patient having an improved heart function, the method comprising the steps of: providing an individual having a cardiac pathology, providing the catheter system of the invention disclosed herein, advancing the first tube through the thoracic circulatory system, the thoracic circulatory system selected from the group consisting of the right atrial appendage of the heart, the right atrium, the left atrium, the right ventricle, and the left ventricle of the individual, and chambers thereof, advancing the distal end of the first tube against the inner wall of the chamber, rotating and advancing the first tube and engaging the coil with the tissue of the myocardium whereby the coil attaches to the myocardium and anchoring the system in the myocardium thereby, advancing the second tube, the second tube comprising a perforator, through the first lumen and puncturing the myocardium, advancing the second tube through the first lumen and through the punctured myocardium and penetrating the myocardium to the outer surface of the myocardium, then advancing the intrapericardial electrical catheter across the outer surface of the myocardium to a site on the outer surface of the myocardium, and operating the intrapericardial electrical catheter, thereby treating the individual having a cardiac pathology and that results in the patient having an improved heart function.

The invention also provides a method of using a catheter system to treat an individual having a cardiac pathology and that results in the patient having an improved heart function, the method comprising the steps of: providing an individual having a cardiac pathology, providing the catheter system of the invention disclosed herein, advancing the first tube through the thoracic circulatory system, the thoracic circulatory system selected from the group consisting of the superior vena cava, the inferior vena cava, the coronary sinus, advancing the distal end of the first tube against the inner wall of the chamber, advancing the second tube, the second tube further comprising a perforator, through the first lumen and puncturing the myocardium, advancing the second tube through the first lumen and the punctured myocardium and penetrating the myocardium to the outer surface of the myocardium, then advancing the intrapericardial electrical catheter across the outer surface of the myocardium to a site on the outer surface of the myocardium, and operating the intrapericardial electrical catheter, thereby treating the individual having a cardiac pathology and that results in the patient having an improved heart function.

In another embodiment the method further comprises a step of flexing the first tube upon entrance into the vasculature to help assist in placement of the catheter. In a still further embodiment, the method further comprises a step of removing the perforator from the thoracic circulatory system. In a yet further embodiment, the method further comprises a step of advancing at least one guidewire through the second tube, and wherein the guidewire is selected from the group consisting of a 0.035″ guidewire and a 0.014″ guidewire and the guidewire advanced through the second tube is a 0.035″ guidewire, further comprising the steps of advancing a sheath over the 0.035″ guidewire, the sheath having a lumen, removing the 0.035″ guidewire, advancing a 0.014″ guidewire through the lumen of the sheath, and removing the sheath. Other guidewires known in the art may also be used in conjunction with the methods and devices disclosed in this patent application.

The invention provides a method of using a catheter system to treat an individual having a cardiac pathology and that results in the patient having improved heart function the method comprising the steps of: (i) providing an individual having a cardiac pathology (ii) providing a catheter system comprising: a first tube, the first tube having a proximal end and a distal end, and defining a first lumen within the longitudinal axis of the first catheter, attaching means having a proximal end and a distal end, wherein the proximal end of the attaching means is affixed to the first tube at or near the distal end of the first tube and the distal end of the attaching means is adapted for attaching to the wall of the mammalian organ, a second catheter, the second tube having a distal end adapted to puncture the wall of the mammalian organ and having a proximal end, defining a second lumen within the longitudinal axis of the second tube, an intrapericardial electrical catheter having a distal end and a proximal end, defining a third lumen within the longitudinal axis of the intrapericardial electrical catheter, wherein the intrapericardial electrical catheter is adapted for insertion through the first lumen and wherein the distal end of the intrapericardial electrical catheter is shaped and adapted for placement against a site upon the surface of the mammalian organ, (iii) advancing the first tube through the superior vena cava, the right atrium, and the right ventricle of the individual, (iv) advancing the distal end of the first tube against the inner wall of the right ventricle, (v) rotating and advancing the first tube and engaging the coil with the tissue of the myocardium whereby the coil attaches to the myocardium and anchoring the system in the myocardium thereby, (vi) advancing the second tube through the first lumen and puncturing the myocardium, (vii) advancing the second tube through the punctured myocardium and penetrating the myocardium to the outer surface of the myocardium, (ix) advancing the intrapericardial electrical catheter through the perforation created and advancing the intrapericardial electrical catheter across the outer surface of the myocardium to a predetermined target site on the outer surface of the myocardium, and (xii) operating the intrapericardial electrical catheter, thereby treating the individual having cardiac pathology and that results in improved heart function.

### BRIEF DESCRIPTION OF THE DRAWINGS

FIG. 1 is a schematic longitudinal cross-sectional representation of one embodiment of the catheter system (7).

FIG. 2 is a schematic transverse cross-sectional representation of the proximal end of the catheter system.

FIG. 3 shows an alternative embodiment of the distal end of the catheter system showing a balloon (9) inflated to guide the catheter to the apex of the left ventricle.

FIG. 4 shows an alternative embodiment of the electrode (5) and attaching means on the distal end of the catheter.

FIG. 5 shows an alternative embodiment of the distal end of the catheter system.

FIG. 6 shows a detail of the catheter system showing successive stages of a method of using the catheter system.

FIG. 7 illustrates a detail of an alternative embodiment of the invention.

FIG. 8 through FIG. 16 show different embodiments of the catheter system of the invention in use.

FIG. 16 shows a detail of the distal end of the catheter system of one embodiment of the invention.

FIG. 17 shows a detail of the distal end of the catheter system of one alternative embodiment of the invention.

FIG. 18 shows a detail of the distal end of the catheter system of another alternative embodiment of the invention.

FIGS. 19 through 21 illustrate several embodiments of the invention and how to use them.

FIG. 22 illustrates a detail of a cross-sectional view of a device of the invention placed in the lumen (34) of a blood vessel.

FIG. 23 shows a detail of an alternative embodiment of the device placed in a blood vessel.

FIG. 24 shows a detail of another embodiment of the device placed in a blood vessel.

FIG. 25 shows a detail of another embodiment of the catheter device in use.

FIG. 26 shows a detail of a cross section of another embodiment of the catheter device comprising a vacuum steering platform (40) being deployed in use.

FIG. 27 illustrates a detail of another embodiment of the invention.

FIG. 28 illustrates another embodiment of the invention.

FIG. 29 illustrates another embodiment of the attaching means disposed on the distal end of the intrapericardial electrical catheter

FIG. 30 illustrates a method of using an adhesive to attach the intrapericardial electrical catheter to the wall of the heart.

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a” “an”, and “the” include plural reference unless the context clearly dictates otherwise. Thus, for example, a reference to “a lead” includes a plurality of such leads, and a reference to “an electrode” is a reference to one or more electrodes and equivalents thereof, and so forth.

One embodiment of the catheter systems comprises an intrapericardial electrical catheter, means for attaching or securing the intrapericardial electrical catheter to the surface of the myocardium, a right ventricular pacing lead, and an attaching catheter comprising means for fixing or anchoring the right ventricular pacing lead to the inner surface of the wall of the right ventricle.

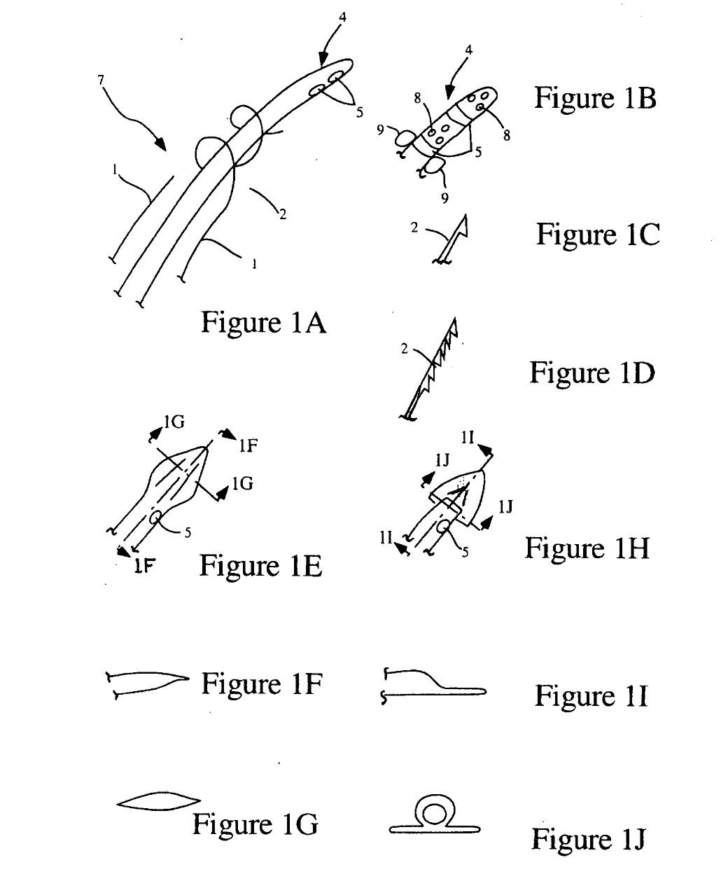

An exemplary catheter system (7) is shown in FIG. 1A; the intrapericardial electrical catheter (4) has at least one first electrode (5), preferably two electrodes, at or near the tip of the distal end of the lead. The tip further comprises a first attaching or securing means (8), such as a stylet, a hook, a clip, a staple, an adhesive, a coil, a barb, a serrated blade or knife, a threaded screw, a vacuum device, or the like. The intrapericardial electrical catheter is concentrically deployed within the lumen of a first catheter or tube, the catheter comprising the right ventricular pacing (RV) lead (3) and at least one second electrode (30). The first tube further comprises at least one second attaching or anchoring means (2). Examples of such second attaching means are, but not limited to stylet, a hook, a clip, a staple, an adhesive, a coil, a barb, a serrated blade or knife, a threaded screw, a vacuum device, or the like. Examples of attaching means are shown in FIGS. 1A, 1B (8), 1C, 1D, 4A, 4B, 5A, 5B, 26A, and 27.

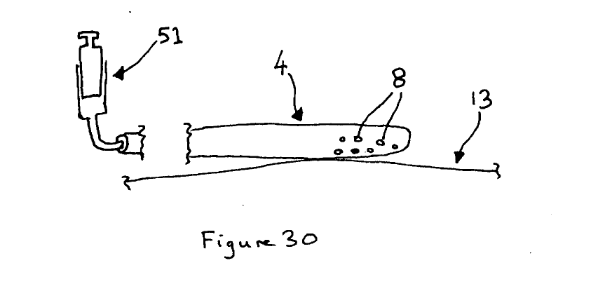

In one example, the attaching or anchoring means is a coil made from a memory-metal alloy, such as NiTi alloy (for example, NITINOL), that is confined as a straightened shape in a lumen within the catheter system. The coil or hook is then advanced through the lumen by an operator and emerges from the distal end of the catheter system, whereby it reverts to the coil or hook shape. The operator then rotates the intrapericardial electrical catheter that results in the coil or hook drilling into the surface tissue of the myocardium and securing or anchoring the intrapericardial electrical catheter at the target site. (See FIGS. 4A and 4B, FIGS. 29A and 29B.)

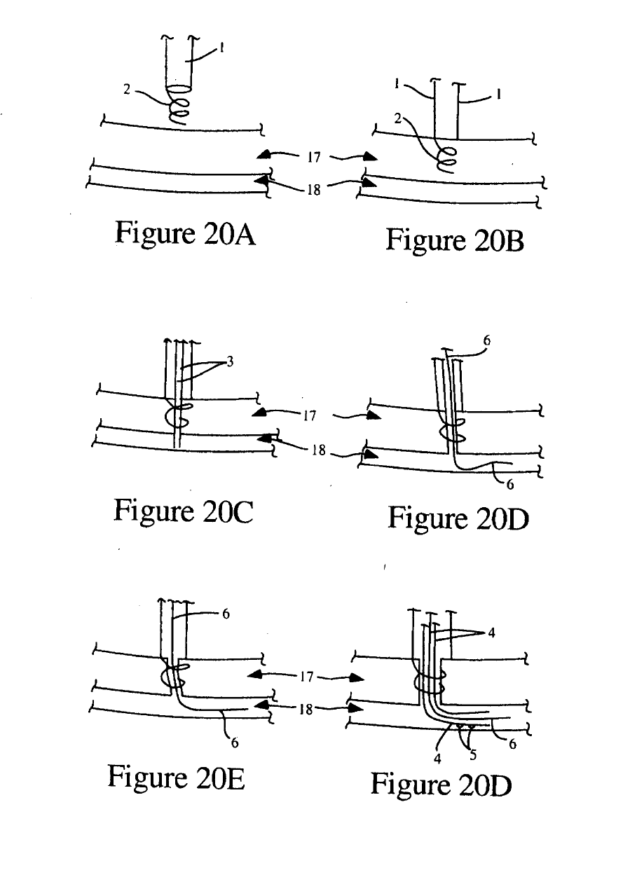

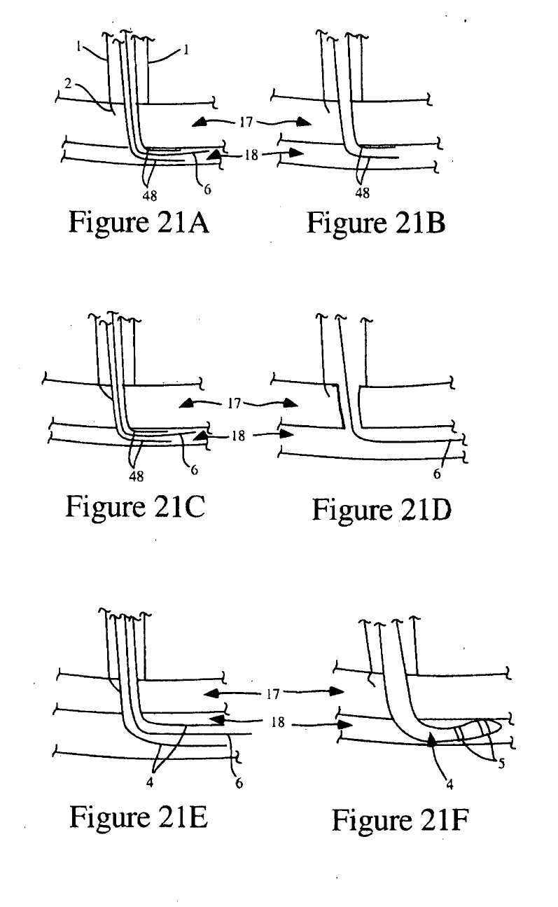

The catheter system can optionally comprise a guide-wire (6), the guide-wire being shaped and adapted for directing the intrapericardial electrical catheter upon leaving the myocardium and flexing the intrapericardial electrical catheter to contort and migrate through the pericardial space until a target area is reached (see FIG. 20 and FIG. 21). The guide-wire can be made of a material such as stainless steel, titanium, a nickel-titanium alloy, or the like that can be shaped and adapted for placement in the myocardium and pericardial space.

The tip of the intrapericardial electrical catheter may be shaped and adapted for dynamic movement within the pericardial space, the interstitial space between the pericardium and the epicardium. The tip may be a blunt bulb as shown in FIG. 1A and FIG. 1B, a diamond-shape as shown in FIG. 1E, an arrowhead as shown in FIG. 1H, or the like. The tip can comprise an inflatable bouyancy balloon, such as shown in FIG. 3, whereby inflation of the bouyancy balloon within the pericardial space assists the operator navigate in the pericardial space (external to the epicardium). The bouyancy balloon may be inflated using fluids or gasses well known in the art, such as, but not limited to, air, nitrogen, helium, water, saline, and the like.

The bouyancy balloon may include a fluorescent or radio-opaque material as part of its structure. Such markers may be placed at various positions on the balloon or manufactured into the fabric of the balloon such that the position of the balloon may be easily visualized by fluoroscopy or other means during surgical procedures in the catheter laboratory (“cath lab”). U.S. Pat. No. 6,599,448 discloses a radio-opaque composition including a polymer or monomer that could be used with the present device. Position of the catheter tip can be seen using a radiopaque marker at its tips. The catheter may have a lumen through which contrast may be injected to visualize location within the intrapericardial space.

The distal end of the intrapericardial electrical catheter may also comprise an alternative first attaching means that attach or secure the distal end of the intrapericardial electrical catheter to the surface (13) of the heart myocardium, as shown in FIG. 5. In this example, the alternative attaching means comprises a vacuum catheter (10) having an outer wall and an inner wall defining a vacuum lumen (43), a proximal end, a distal end, the inner walls of the vacuum catheter further defining a lumen (12), the lumen having the distal end of the intrapericardial electrical catheter (4) circumferentially disposed within. One face of the outer wall at or near the distal end of the vacuum catheter is flat and comprises a plurality of apertures (11). The proximal end of the vacuum catheter is in fluid communication with a vacuum pump. In use, the distal end of the vacuum catheter is advanced by an operator to the target site on the surface of the myocardium, the vacuum pump is switched on, thereby decreasing the internal gas pressure within the catheter vacuum lumen and resulting in air entering through the apertures (11) from between the flat face of the vacuum catheter and near the surface of the myocardium (13), thereby resulting in a vacuum between the flat face and the surface of the myocardium and the distal end of the vacuum catheter becomes secured by suction in close proximity to the target site. It helps the catheter to maintain its position along the surface of the myocardium. One or more of the devices disclosed herein may be attached for a short time or permanently to the anatomy. One or more of the devices disclosed herein may be removed after performing a diagnostic, therapeutic, or preventative procedure.

The distal end of the intrapericardial electrical catheter can further comprise devices for attaching to the myocardium surface. Such attaching devices can be a jaw hook, an adhesive or glue, a balloon, a pedal, or the like. In one embodiment, the intrapericardial electrical catheter does not comprise a lumen. In another embodiment, the intrapericardial electrical catheter is pre-shaped.

Adhesive means for surgical use are well known to those of skill in the art, and are for example biocompatible adhesives including, but not limited to, cyanoacrylate adhesives, fibrin sealants, chemically-modified natural proteins such as, but not limited to, collagen or albumin further comprising aldehyde cross-linking agents such as, but nor limited to, glutaraldehyde or formaldehyde, gelating-resorcinol-formol glues, and the like.

Advantages of using a biocompatible glue or a foam or any adhesive, for example are that there is expected to be no necrosis of the myocardial tissue. Necrosis is caused by pressure cutting off the blood supply of the capillary beds under the contact point. Another advantage is that there can be little or no migration of the device medially or laterally with respect to the surface of the myocardium. A considerable pulling force would be required or expected to dislodge the device so positioned. A further advantage of using a biocompatible glue or foam or any adhesive can be that the myocardium dynamics are unaltered.

The biocompatible glue may be delivered by, for example, a porous material that is part of the outer edge of the device and the glue may be pumped out from within the device. In another alternative, a breakable capsule containing the glue is in the device and the capsule is broken remotely by an operator. Glue can also be delivered via tubing from outside the individual. The device may comprise an adhesive outer surface, for example, a sticker comprising an adhesive and a protective material whereby the protective material is peeled away to reveal the adhesive surface.

In another embodiment, the distal end of the second catheter can be beveled, the angle of the bevel relative to the longitudinal axis of the catheter being between about 0 and 90 degrees, between about 15 and 75 degrees, or between about 30 and 60 degrees, thereby enabling an operator to remotely advance the catheter into and puncture the wall of the heart or a blood vessel under conditions when the catheter system is non-perpendicular to the wall of the heart or a blood vessel.

The first tube can further comprise a gasket ring, the gasket ring being disposed at or near the distal end of the first tube and having a diameter greater that the diameter of the first tube and that provides an impeding element to impede and prevent the first tube from advancing further through the myocardium. The gasket ring can be positioned at about between 1 mm and 5 mm from the distal end of the first tube, preferably a distance equal to about the thickness of the myocardium. Such myocardium thickness can vary between individual and therefore the distance can be adjusted by the operator or alternatively, a number of different first tube distal ends with differing proportions can be manufactured and thereafter selected by the operator. In one method embodiment, the first tube is purposefully advanced through a heart wall or through the thoracic vasculature such that the distal tip of the first tube is located in the intrapericardial space.

The second tube can further comprise a gasket ring, the gasket ring being disposed at or near the distal end of the second tube and having a diameter greater that the diameter of the second tube and that provides an impeding element to impede and prevent the second tube from advancing further through the myocardium. The gasket ring can be positioned at about between 1 mm and 5 mm from the distal end of the second tube, preferably a distance equal to about the thickness of the myocardium. Such myocardium thickness can vary between individual and therefore the distance can be adjusted by the operator or alternatively, a number of different first tube distal ends with differing proportions can be manufactured and thereafter selected by the operator.

The first tube and the intrapericardial catheter can comprise a plurality of electrodes, the electrodes positioned on the outer or the inner surface of the first tube and/or catheter at regular intervals. Each electrode can be controlled independently of another electrode thereby enabling an operator to choose which electrode in operation achieves the most desirable result during pacing. The electrodes can also be used as sensors to detect changes in heart activity prior to, during, and/or after implantation of the RV lead and/or intrapericardial electrical catheter. Electrodes or coils that are used for defibrillation can also be positioned on the external surface of the first tube, of the second tube, and/or of the intrapericardial electrical catheter.

The second tube and the intrapericardial catheter can comprise a plurality of electrodes, the electrodes positioned on the outer or the inner surface of the second tube and/or catheter at regular intervals. Each electrode can be controlled independently of another electrode thereby enabling an operator to choose which electrode in operation achieves the most desirable result during pacing. The electrodes can also be used as sensors to detect changes in heart activity prior to, during, and/or after implantation of the RV lead and/or intrapericardial electrical catheter.

The catheter systems of the invention are used to implant at least one intrapericardial electrical catheter epicardially via the pericardial space and use the intrapericardial electrical catheter lead as a guide-wire to introduce a right ventricular pacing lead into a suitable position in the right ventricle. In this instance the intrapericardial electrical catheter is placed via puncture of the right ventricle. Then the RV lead is introduced using a separate catheter system. Alternatively, the intrapericardial electrical catheter may be placed into the pericardial space by accessing the pericardial space through the inferior vena cava (IVC), the superior vena cave (SVC), the right atrial appendage of the heart (RAA), the right atrium (RA), and/or the right ventricle (RV). Optionally, the intrapericardial electrical catheter can be introduced to the pericardial space through the left atrium (LA), the left ventricle (LV), the coronary sinus (CS), the coronary vasculature (CV), or the femoral artery (FA) via the aorta; Alternatively, the pacing leads may be implanted using a laparoscope to access any internal blood vessel or organ of the mammalian anatomy. The catheter system is torquable, the control and maneuverability of the system components are simple to operate, and may be operated by an operator having less expertise than a physician.

Other medical devices can be delivered using the catheter system disclosed herein, such as, devices for pacing one or more heart chambers (LV, RV, right atrium, left atrium, etc.), defibrillating or more heart chambers (LV, RV, right atrium, left atrium, etc.), ablating a region of the heart or the surrounding vasculature, injecting a medication or biological materials such as stem cells, diagnosing a source of arrhythmia, introducing or withdrawing fluids, introducing substances or devices that prevent pathological expansion or dilation of heart tissue, accessing the coronary vasculature to perform a diagnostic or therapeutic procedure, measuring electrical properties of the heart, or the like The intrapericardial electrical catheter can further comprise a catheter selected from the group consisting of an ablation catheter, a cryogenic catheter, a drug-delivery catheter, and a cell-delivering catheter or the like. Delivery systems for delivering drugs, hormones, growth factors, cytokines, chemokines, cells (for example cardiomyocytes, adipocytes, primary endothelial cells, primary nerve cells, and/or stem cells), or the like, are well known to those of skill in the art.

The catheter system can further comprise a plurality of electrodes disposed upon the surface of the intrapericardial electrical catheter the electrodes comprising an electrical device selected from the group consisting of a defibrillator coil, a pacing electrode, and/or a sensing electrode.

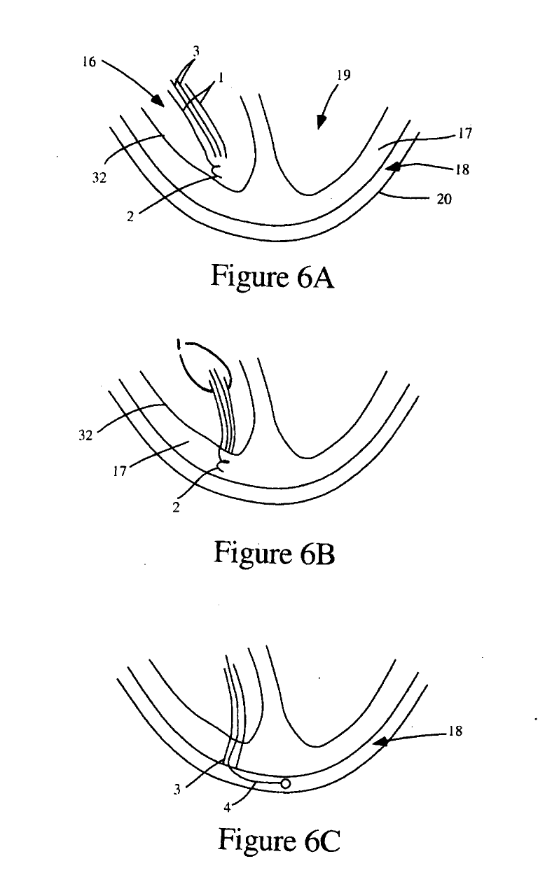

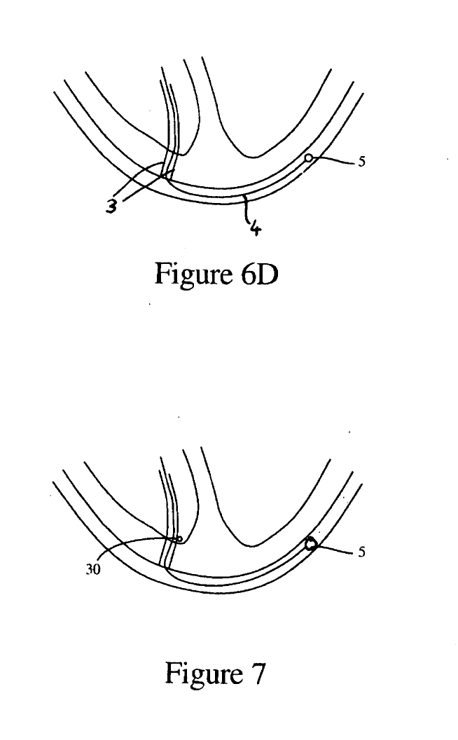

In use, the operator makes an incision in the skin of the individual to be treated for cardiac pathology near a blood vein. The operator then advances the first tube (1) through the venous system into the right atrium (RA), the tricuspid valve, and then into the right ventricle (RV; 16) towards the apex of the heart. (See FIG. 6A.)

A cardiac pathology is, for example, but not limited to, congestive heart failure (CHF), arrhythmias (supraventricular and ventricular), a viral infection, delivery of therapeutic compositions, or the like.

This first tube comprises a second attaching or anchoring means (2) that is advanced towards the apex of the heart. The RV catheter has flexing ability to help maneuver it within the confines of the heart and the vasculature. The operator guides the catheter system by observing the position of a suitable marker at or near the distal end of the first tube. The marker can be, but is not limited to, a dye, a radio-opaque material, a magnet, an ion source, or the like.

The operator then advances the distal end of the catheter system against the inner surface (32) of the RV and engages the wall of the myocardium (17). If the second attaching or anchoring means is a coil or a threaded screw, the operator rotates the catheter system about the longitudinal midline and advances the catheter system through the circulatory system and thereby attaching to the surface (32) of the myocardium (17). (See FIG. 6B.) The attaching means, for example a coil, can also comprise at least one electrode.

The operator advances the intrapericardial electrical catheter through the surface of and into the myocardium thereby attaching to the myocardium. The distal end of the second tube (3) may be shaped and adapted to comprise puncturing or cutting means so that puncturing and penetrating the myocardium is performed without causing damage to the immediate surrounding tissues. Such puncturing or cutting means can be, but are not limited to, a needle, a serrated edge, a blade, an electrocauterizing device, a guidewire or the like.

The intrapericardial electrical catheter is advanced though the myocardium until the distal end of the intrapericardial electrical catheter emerges at the outer surface of the myocardium. The operator then advances the intrapericardial electrical catheter until it emerges from the distal end of the first tube into the pericardial space (18). (See FIG. 6C.)

The RV lead is made from a material that is flexible and that may be controllably flexed so that the tip of the RV lead does not damage the intracardiac structure and tissue. The intrapericardial electrical catheter can comprise a material that can comprise a memory-metal alloy that reverts to a curved shape upon emerging from the constraints of the first lumen. The material can comprise a flexible material with plasticity sufficient to be deformed when a guide-wire (6) is introduced into the inner lumen of the intrapericardial electrical catheter. The guide-wire may be remotely flexed by the operator using means well known in the art, such as disclosed in U.S. Pat. No. 5,397,321 that describes a catheter having a steering wire for transmitting bending (torquing) force to the catheter from a remote control mechanism.

The first (RV) tube can further comprise a vacuum chamber and the distal end of the second catheter can further comprise a vacuum seal and a penetrating needle. In use, the operator may advance the first tube through the circulatory system and position the distal end and vacuum seal against the wall of the heart or blood vessel. The proximal end of the first tube can be in fluid communication with a vacuum pump, wherein upon creating a vacuum in the vacuum chamber, the distal end of the first tube becomes secured by suction in close proximity to the target site upon the wall of the heart or blood vessel, and oriented such that the longitudinal axis of the first tube is approximately perpendicular to the wall of the heart or blood vessel. This embodiment has particular advantages when using a penetrating needle, as a large amount of force is not required to cause the needle to penetrate the wall of the heart or blood vessel. Instead, the wall of the heart or blood vessel is drawn down into the suction face of the catheter system, causing it to become impaled upon the needle, which then may be advanced through the wall of the heart or blood vessel using minimal force.

The distal portion of the sheath of the catheter system can be curved. The distal portion can be located between 1 and 20 mm from the distal end of the sheath and can be about between 1 and 5 mm in length. The curvature must be sufficient to facilitate proper positioning of the distal end of the intrapericardial electrical catheter within the pericardial space, so that it is directed away from the pericardium and brought close to a target site. The curve of the distal portion of the sheath may describe a total angle of between 0 and 180 degrees, or between 5 and 90 degrees or between 55 and 90 degrees, or preferably between about 70 and 90 degrees. Such curvatures are approximate and the curvature may be adjusted in order to fit a particular purpose or anatomy. The distal portion of the sheath can comprise a material that is substantially soft to allow navigation in the intrapericardial space.

The operator may then advance the intrapericardial electrical catheter within the pericardial space (18) towards the target site and position the intrapericardial electrical catheter and electrode (5) at or near the desired pacing location (see FIG. 6C and FIG. 6D).

The first catheter may comprise the RV lead and second electrode (30). The second electrode is integrated into the RV lead such that when the first tube is attached or anchored to wall (32) of the myocardium, the second electrode is positioned at the surface of the inner wall of the myocardium (see FIG. 7). The operator passes an electrical current between the two electrode poles of about 2V at 0.5 ms. More preferably the energy is about 1.5 V at 0.5 ms, 1.0 V at 0.5 ms, or 0.5 V at 0.5 ms. The operator is then able to pass a series of test electrical currents through the RV lead and thereby determine the current passing between the first electrode and the second electrode through the myocardial tissue. The current may be between about 0.5 mA and 10 mA. More preferably, the current is between about 2 mA and 7 mA. Most preferable is a current of about 2 mA. The operator engages the attaching or securing means to the myocardium thereby attaching or anchoring the intrapericardial electrical catheter to the myocardium.

The first catheter attaching mechanism may be an exposed helical coil that is protected from the intracardiac tissues with a dissolving mannitol or coating or other biodegradable coating. In another embodiment, the coil is protected by a sheath that protrudes from the distal region of the coil.

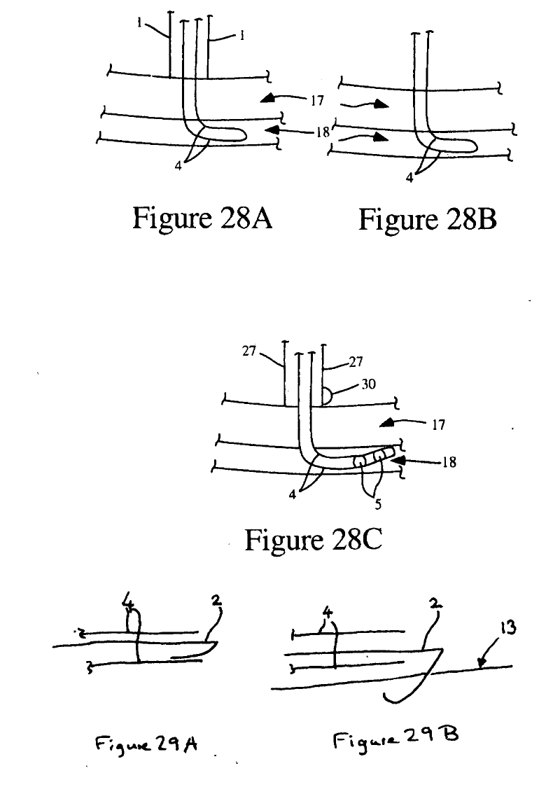

Optionally, the operator may retract and remove the first tube (1) and insert another second tube comprising an RV lead over the outer surface of the intrapericardial electrical catheter thereby using the intrapericardial electrical catheter as a rail to guide the RV lead to a position against the inner wall of the RV (see FIG. 28C).

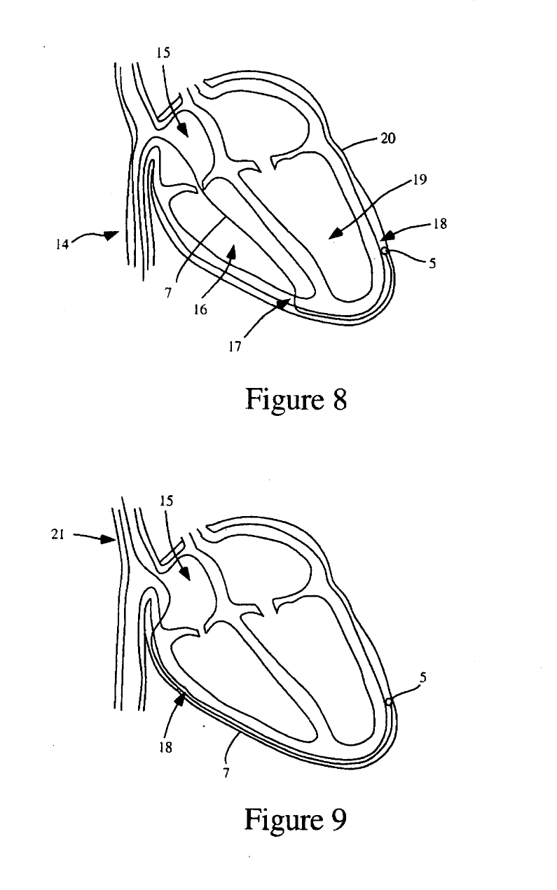

The catheter system of the invention may be directed to the site on the surface of the myocardium of the left ventricle through a number of different anatomical access routes. Several such routes are illustrated by example on FIGS. 8 through 15.

The catheter system may be threaded through the IVC (14), the RA (15), the RV (16) and then penetrates the myocardium near the apex of the heart. The intrapericardial electrical catheter comprising an electrode (5) is then threaded through the pericardial space (18) under the pericardium (20) to the site. (See FIG. 8.)

The catheter system may be treaded through the SVC (21), the RA, and then penetrates the myocardium wall of the RA. The intrapericardial electrical catheter is then threaded through the pericardial space (18) under the pericardium to the site. (See FIG. 9.)

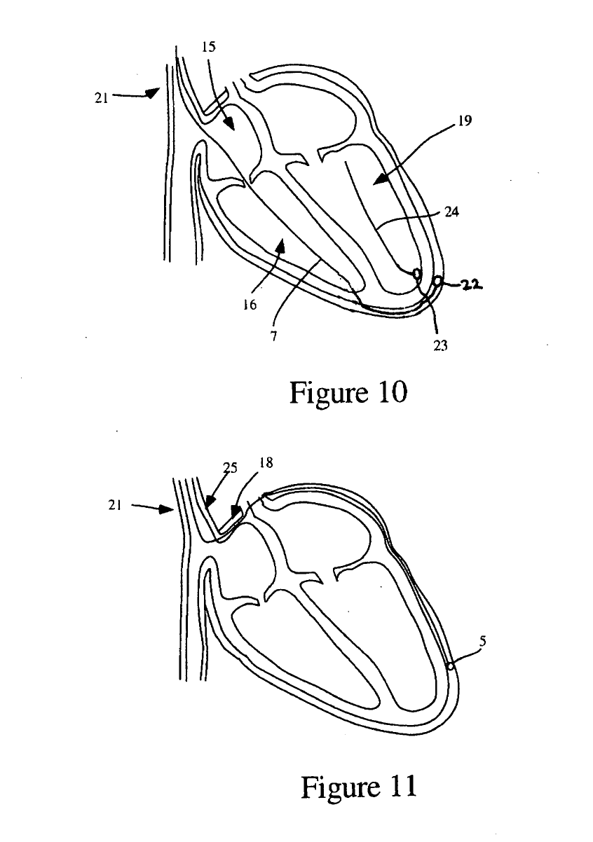

The catheter system may be treaded through the SVC (21), the RA, the tricuspid valve, and the RA. In this example, the tip of the catheter system comprises the intrapericardial electrical catheter and a magnet (22). A deploying catheter (24) comprising an electromagnet (23) is introduced into the circulatory system through the femoral artery and then threaded through the aorta into the LV (19). The intrapericardial electrical catheter is advanced through the RV, the myocardium, and into the pericardial space. The electromagnet is activated thereby attracting the magnet at the tip of the intrapericardial electrical catheter towards the electromagnet within the heart chamber. The operator positions the electromagnet such that the magnetic tip of the intrapericardial electrical catheter is against the surface of the heart and can then be activated for pacing (See FIG. 10.)

In another alternative, the catheter system may be treaded through the SVC (21) and the cardiac vein and then punctures and penetrates the endothelium of the cardiac vein, thereby accessing the pericardial space (18). The intrapericardial electrical catheter is then threaded under the pericardium to the site. (See FIG. 11.)

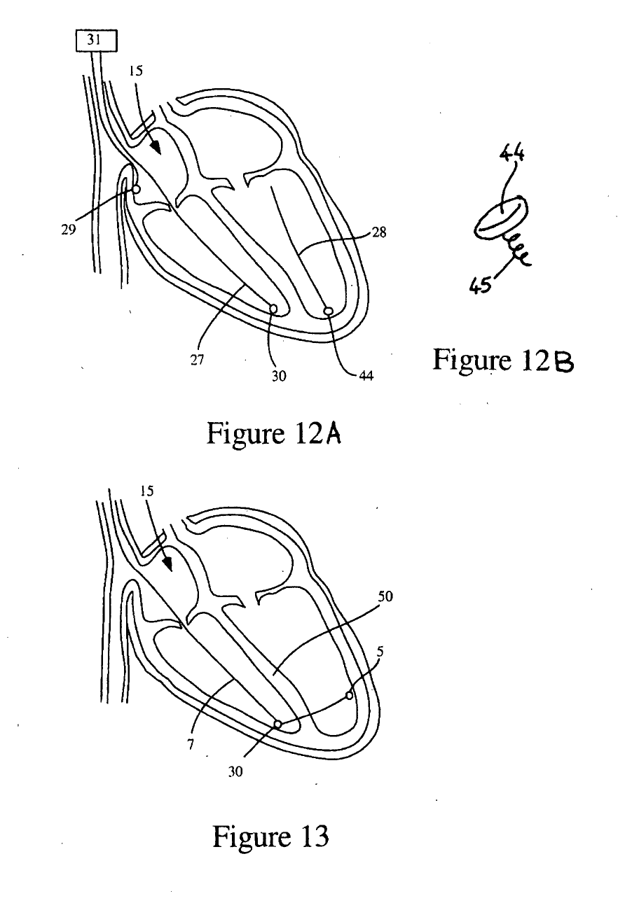

In still another alternative, the catheter system can further comprise a right atrial pacing (RA) lead (26) and wherein the distal end of the RA lead comprises at least one electrode (29). The RA lead is inserted through the vena cava using a catheter and the distal end of the RA lead comprising the electrode (29) is positioned against the wall of the right atrium (15). The RV lead (27) is inserted through the vena cava using a catheter and the distal end of the RV lead comprising an RV electrode (30) is positioned against the wall of the right ventricle. A single catheter may be used to insert and position both the RA lead and the RV lead, or, alternatively, a plurality of separate catheters may be used to insert the RA lead and RV lead. The RA lead and the RV lead are in electrical communication with a pacemaker (31). A catheter (28) is introduced into the circulatory system through the femoral artery and then threaded through the aorta into the LV. The catheter (28) comprises an LV pacer (44) that comprises attaching or securing means, such as those described above. The LV pacer is attached or secured to the inner wall of the LV by the operator using remote controlling means, such as a wire, a ribbon, or the like. Operation of the LV pacer is induced using the pacemaker/RA lead/RV lead system. The LV pacing catheter (28) can be retracted and the LV pacer remain positioned and secured in the wall of the heart (See FIG. 12A and FIG. 12B)

A preferred embodiment of the invention is illustrated on FIG. 13. The catheter system (7) comprises at least one RA electrode (30) and at least one LV electrode (5). The catheter system is introduced through the SVC and into the RA (15). The catheter system is then deployed against the wall of the RV, the operator uses the puncturing or cutting means of the catheter system to puncture and penetrate the wall thereby accessing the pericardial space and the operator advance the intrapericardial electrical catheter to the target site. In an alternative embodiment, the catheter system is introduced into the heart through the IVC.

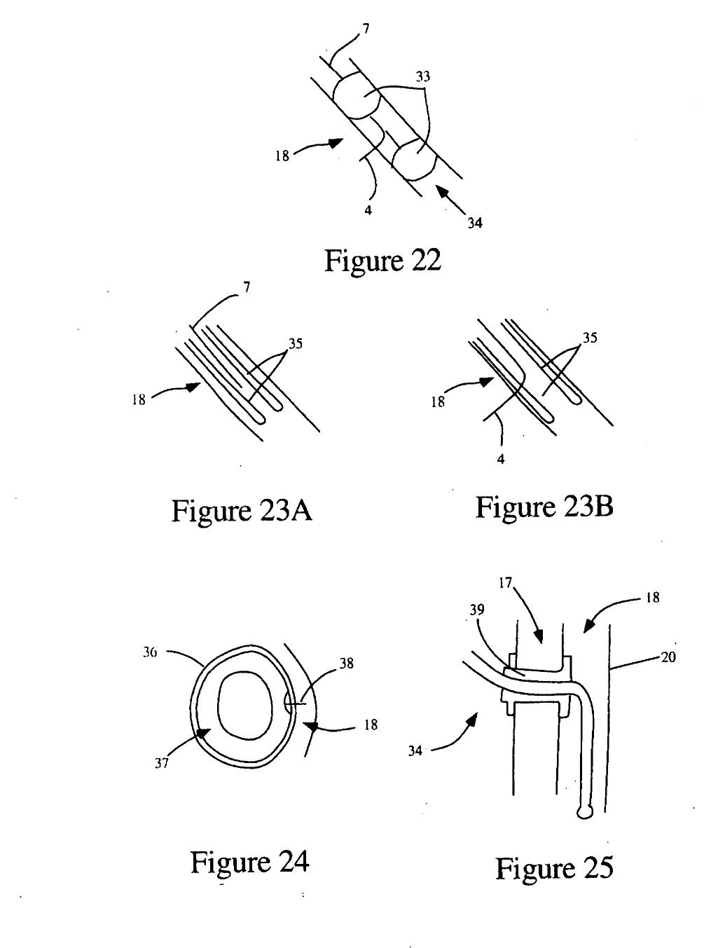

In yet another alternative, the catheter system may be threaded through the SVC the RA, and the coronary sinus and then secured in place using fixing or anchoring means. Such fixing or anchoring means can be, for example, a balloon, an expandable sheet, an expandable cuff, an expandable sealing ring, or the like. The fixing or anchoring means are deployed within the confines of the coronary sinus thereby wedging or anchoring the catheter system within the lumen of the coronary sinus. A second catheter (4) comprising puncturing and/or penetrating means is deployed by an operator and the operator advances the second catheter thereby puncturing and penetrating the wall of the coronary sinus and accessing the pericardial space. Puncturing and/or penetrating means can be, but are not limited to, a needle (38), a blade, a serrated edge, an electrocauterizing device, or the like. Examples of fixing or anchoring means and how they may be used are illustrated in FIG. 22, FIG. 23, FIG. 24, and FIG. 29.

The catheter system of the invention can also comprise a valve (39) that is shaped and adapted for positioning and securing the system within the myocardium (17) or the wall of a blood vessel. The valve (39) is fixedly attached to the surface of the first catheter or the intrapericardial electrical catheter and forms a seal between the outer surface of the first catheter or the LV lead and the region of puncture in the wall of the heart. The valve so positioned may prevent tamponade during the accessing of the pericardial space. (See FIG. 25.)

Additional devices can be included in the catheter system that may assist sealing the puncture and passage created in the wall of the heart or blood vessel. For example, as illustrated in FIG. 27, the distal portion of the second catheter can comprise a sleeve (41) having barbs (42) shaped and adapted for placement in the puncture and/or passage to anchor the distal end of the second catheter within the myocardium. Similarly, the sleeve can comprise a thread structure that can be remotely rotated and thereby screw into the wall of the heart myocardium and thereby anchor the catheter system in position. The thread structure has the advantage of being rotateable in the opposite direction, thereby enabling an operator to remove the catheter system from the wall of the heart or blood vessel.

FIG. 27 also illustrates an additional optional element of the sleeve, the aperture at the distal end of the sleeve being situated asymmetrically and facing at an angle of between 45 and 90 degrees to the longitudinal axis of the sleeve. The advantage of this structure is that the intrapericardial electrical catheter, upon being advanced through the aperture, is forcibly directed in a path that is generally parallel to the surface of the myocardium and the pericardium, thereby reducing risk of perforating the pericardium. This structure and other structures that execute the same ends have the further and additional advantage of enabling the operator to direct the progress and path of the LV lead over or near to the surface of the myocardium in individuals who have no pericardial tissue, such as patients having undergone bypass surgery. The term “intrapericardial space” is to be interpreted broadly as the region between pericardium and a structure, for example, a heart wall enclosed by the pericardium, or the region around the heart in patients who have no or little parietal pericardium.

The catheter system can comprise several devices that may be used to advance and/or move the intrapericardial electrical catheter through the pericardial space. For example, the tip of the intrapericardial electrical catheter can comprise a blade-like shape (FIGS. 1E, 1F, and 1G) or an arrowhead-shape (FIGS. 1H, 1I, and 1J) that may dynamically pass through the pericardial space between the myocardium and the pericardium by virtue of their shape without resulting in excessive injury to the surrounding tissue. The tip can comprise a balloon (FIG. 3) that has buoyancy, thereby allowing an operator to more easily control, navigate, and steer the intrapericardial electrical catheter within the confines of the pericardial space. The tip or the distal portion of the intrapericardial electrical catheter can further comprise a bellows device that has accordion-like folds or pleats, whereby a remote operator can sequentially expand and contract the bellows thereby either advancing or, if desired, retreating, the distal portion of the intrapericardial electrical catheter through the pericardial space.

The intrapericardial electrical catheter can comprise a vehicle that is shaped and adapted for placement within the pericardial space. The vehicle comprises at least one wheel or track mechanism, the wheel of track mechanism being steerable using guide-wire by a remote operator. The intrapericardial electrical catheter can further comprise a pair of steerable “wings” that comprise a plurality of rudder elements disposed upon one or more surfaces and guide-wire steering elements. The operator can adjust the angle of the “wings” relative to the intrapericardial electrical catheter using the guide-wire and the rudder elements may tend to direct the intrapericardial electrical catheter perpendicular to that angle.

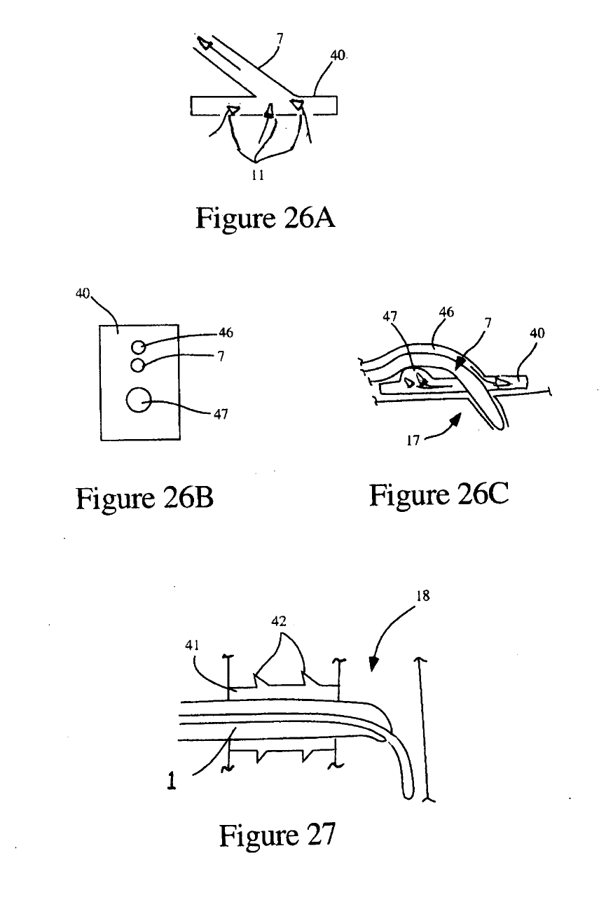

The catheter system can comprise a negative/positive pressure steering platform, as exemplified in FIG. 26. The distal end of the catheter system is attached to the negative/positive pressure steering platform using a flexible joint, comprising a substantially deformable materials such as latex rubber, polymeric compositions, or the like, thereby enabling the negative/positive pressure steering platform a considerable range of motion relative to the distal end of the catheter. As shown in FIG. 26A, air is drawn up through a lumen of the catheter system (7) through apertures (11) in the negative/positive pressure platform (40). The face of the negative/positive pressure steering platform is positioned against the surface of the heart and the movement of air creates a vacuum seal between the face of the negative/positive pressure steering platform and the surface of the heart, thereby allowing the operator to maintain the distal end of the catheter in a desired fixed position.

In an alternative embodiment, as shown in FIG. 26B and FIG. 26C, the negative/positive pressure platform can comprise an inflatable balloon (47) that, when inflated during use by pumping air or the like through a lumen (46) into the negative/positive pressure platform, exerts a force upon the catheter system (7) causing the catheter system to contort and alter its position relative to the negative/positive pressure platform and thereby steers the catheter in a desired direction (Open arrows representing direction of air movement). In another embodiment, the intrapericardial catheter can have an electrocauterizing device at its distal region to help break adhesions and enable easier maneuverability in the intrapericardial space and/or the area outside the epidcardium.

The body of the device (7) may have a diameter of, for example, from 3 mm to 40 mm, or from 3 mm to 35 mm, or for example about 3 mm, 7 mm, 12 mm, 25 mm, 18 mm, 22 mm, 25 mm, 28 mm, 31 mm, 35 mm, or 40 mm. The length of the device may be any length compatible with its function of placing an intrapericardial electrical catheter or other medical device against the surface of the heart, and the device may (or may not) be shorter than the deploying device and/or system that is used to deploy it into the heart. For example, the device may be from 4 cm to 60 cm in length, or for example about 5 cm, 7 cm, 10 cm, 14 cm, 18 cm, 22 cm, 30 cm, 45 cm, or 55 cm in length. The body of the invention may be of variable fixed lengths, or it may be of dynamically adjustable length by use of a telescoping designs. The body of the invention is generally a flat or elongated cylinder, though it may be of any suitable cross-sectional shape such as oval or polygonal. The body of the invention may be rigid or may be flexible. A flexible body is desirable when using a flexible laparoscope.

The catheter systems of the invention are used to access the pericardial space through the SVC, IVC, RA, RV, right atrial appendage, or coronary sinus. In these chambers, the delivery component can be removed and does not require anchoring. It solely acts as a delivery system for access to the pericardial space. Once the pericardial space is accessed a lead, or catheter or medications may be delivered into the space.

The catheter systems of the invention are used to access the pericardial space through the RA, right atrial appendage, RV, LA, LV, CS, femoral artery. In these chambers, the delivery component can or cannot be attached to the walls of the chamber via an anchoring mechanism. Through the anchored device access is obtained in the pericardial space. Once the pericardial space is accessed a lead, or catheter or medications may be delivered into the space.

The catheter systems of the invention are used to implant a pacing lead within a chamber of the heart through which another pacing lead is delivered via a transmyocardial puncture. The initial catheter may be attached to the right atrium, right atrial appendage or right ventricle. Through this catheter a perforating mechanism is used to access the pericardial space. In one embodiment the pacing lead is placed through the lumen of the initial catheter after the perforation has been made and the perforating mechanism has been removed whereby the pacing lead enters the pericardial space directly.

In a second embodiment, a wire is placed through the perforating mechanism into the pericardial space and then the pacing lead is advanced over the wire into position after the perforator is removed.

In a third embodiment a series of wires are placed. Initially, a wire is placed through the perforating mechanism. The perforating mechanism is removed. A sheath is then placed over the wire to maintain access in the pericardial space. Then a second wire is placed through the sheath. The sheath is removed and the lead is placed over the wire.

The catheter placed into the pericardial space can be a pacing lead. The second pacing lead can pace any of the 4 chambers of the heart in the pericardial space.

In another embodiment, the catheter placed in the pericardial space is an ablation catheter using radiofrequency energy.

In another embodiment, the catheter placed in the pericardial space is an ablation catheter using cryoablation.

In another embodiment, the catheter placed in the pericardial space delivers medicine.

The first catheter that is placed and attached to the myocardium can have the ability to pace with the distal coil fixation mechanism acting as an electrode.

In another embodiment, there are two separate electrodes for pacing.

In another embodiment there are a plurality of electrodes.

The first catheter that is fixed to the wall of the heart chamber can also be a shocking lead with at least one shocking coil.

The catheter placed in the intrapericardial space can have a shocking component. This coil may provide a lower defibrillation threshold as the energy is truly directed across the heart.

The catheter placed in the intrapericardial space can have a multitude of electrodes for pacing.

The tip of the intrapericardial catheter comprises an attaching means such as stylet, hook, clip, staple, adhesive, coil, barb, serrated blade or knife, threaded screw, vacuum or the like.

The catheter used to deliver the intrapericardial catheter has a flexing mechanism whereby, positioning across valves is made easier.

The perforating mechanism can be a needle with a lumen allowing the transduction of pressure in the needle to help determine access into the intrapericardial space.

The perforating mechanism can be a wire that once positioned in the intrapericardial space can become soft with the removal of an inner stylet.

The perforating mechanism can be a wire that at the time of penetration through the vasculature is hard. Once the perforation is completed with pulling the wire, the means by which it is made stiff are broken and the distal tip becomes soft and allows navigability in the pericardial space.

Once access is obtained in the intrapericardial space, the wire used to navigate can also be a pressure wire allowing measurement of pressure in the needle and across the needle to help assure access in the intrapericardial space.

The perforating mechanism can have the ability to measure impedence which helps determine entrance into the pericardial space.

Once access is obtained in the intrapericardial space, the guidewire used to position the intrapericardial catheter can have a multitude of characteristics. It can comprise a lubricious outer coating to assist with maneuverability. It can be steerable and preshaped.

The intrapericardial catheter can have a Doppler or ultrasound on its distal end to help assess for other vasculature before fixation.

The intrapericardial electrical catheter can have slits or perforations or a region of porous material (such as foam) at its distal region. These slits or perforations or porosity allow for the withdrawal of extra fluid in the intrapericardial space and also give close apposition between the catheter and the myocardium and the pericardium. Suction can be applied prior to the injection of glue. (See FIG. 30.)

In a separate embodiment, suction is applied at the proximal end to assure close apposition of the lead and the epicardium while glue is exuded through the distal end.

In another embodiment the perforations and slits allow the removal of fluid in the case of tamponade.

The intrapericardial lead can measure thoracic impedence and aid in the treatment of heart failure.

Alternatively to that explained above, the intrapericardial electrical catheter can be placed first via a sheath that has the ability to flex. Through the sheath a perforating mechanism is used to perforate the myocardium. Through the perforating mechanism with or without the use of a guidewire, an intrapericardial lead is placed in the intrapericardial space. The intial sheath is removed and the RV lead is placed either over the intrapericardial electrical catheter or to the side of the intrapericardial electrical catheter as in a railing system.

The perforating mechanism may have a curvature whereby once the pericardial space is accessed it will curve along the curvature of the heart and decrease the risk for perforation of the pericardium.

In an alternative embodiment the RV and intrapericardial electrical catheter are one. A non-fixating sheath is delivered to the RV apex. Using a perforating mechanism the RV wall is perforated. A long catheter with a multitude of electrodes is advanced into the intrapericardial space. The catheter is fixed to the epicardium. The sheath is removed. The electrodes are positioned such that those that cross the RV myocardium can be used to pace the RV and those on or near the optimal LV pacing site can be chosen to pace the LV.

In another embodiment, the intrapericardial electrical catheter can have a cutting mechanism used to break adhesions in patient status post coronary artery bypass grafting. The lead may also have a suction mechanism to keep it crawling along the heart surface and not floating in the mediastinal area. The cutting mechanism may be barbs, knife like extrusions, laser, electrocautery and the like.

The outer surface of the devices disclosed herein comprises various arrangements to increase the bond strength of the intrapericardial electrical catheter or another medical device to heart tissue. Examples of such arrangements include but are not limited to:
1. Porous coverings or patches around the medical devices such a foam coverings or patches.
2. Mechanical surface modifications such as surface roughening, creating one or more grooves on the surface.
3. Surface coatings such as hydrophilic coatings.

The structural elements of the invention, for example, tubes, catheters, platforms, sheaths, rings, coils, needles, or the like, disclosed herein can be constructed from a variety of materials including polymers such as silicone, polyurethane, polyethylene, acrylonitrile butadiene stryrene (ABS), polycarbonate, polypropylene, styrene, polyamide (nylon), polyimide, PEEK, PEBAX, polyester, PVC, fluoropolymers (TEFLON), co-polymers. Reinforcement elements such as metallic (stainless steel, NITINOL, chromel) or polymeric braids or coils can be used in construction. Metal and other conductive materials can be used to conduct electrical current along the length of a catheter. These conductive elements could be constructed of stainless steel, copper, gold, platinum, silver, titanium, NITINOL, conductive epoxy, conductive polymers. Elements could be included in construction to make the catheters more visible to x-ray imaging. These elements can include tantalum, platinum, iridium, gold, stainless steel, silver, nickel-titanium alloys, polymer compounding agents such as barium sulfate and titanium oxide. Semiconductor materials can also be used for purposes of sensing. Thermocouples and thermistors can also be used to measure temperature.

The catheter system can be manufactured so as to conform to an individual's own heart, i.e. can be custom-molded. The shape and size of the catheter system is determined using measurements taken from, for example, an electromagnetic scan of the patient's anatomy using imaging technology such as MRI, CAT scans, or the like.

### DETAILED DESCRIPTION OF THE DRAWINGS

FIG. 1 is a schematic longitudinal cross-sectional representation of one embodiment of the catheter system (7). FIG. 1A shows the first tube (1), the second attaching or securing means (2), and the intrapericardial electrical catheter (4) comprising at least one first electrode (5). FIG. 1B shows a detail of the intrapericardial electrical catheter showing an embodiment of the electrodes (5) and the first attaching or securing means (8). In this embodiment, the attaching means is a glue extruded from the lumen of the intrapericardial electrical catheter through apertures in the wall of the intrapericardial electrical catheter. Also shown is an inflatable balloon (9) that can be concentrically disposed around the external wall of the intrapericardial electrical catheter and can be inflated to stabilize the distal end of the catheter prior to and during attaching to the wall of the heart. FIGS. 1C and D show two further embodiment of the second attaching or securing means. FIGS. 1E, 1F, and IG show an alternative embodiment of the tip of the intrapericardial electrical catheter in view from above (FIG. 1E), from the side (FIG. 1F), and in cross-section (FIG. 1G). FIGS. 1H, 1I, and 1J show an alternative embodiment of the tip of the intrapericardial electrical catheter in view from above (FIG. 1H), from the side (FIG. 1I), and in cross-section (FIG. 1J). (Interrupted lines show the plane of the cross-section shown in the indicated Figure.

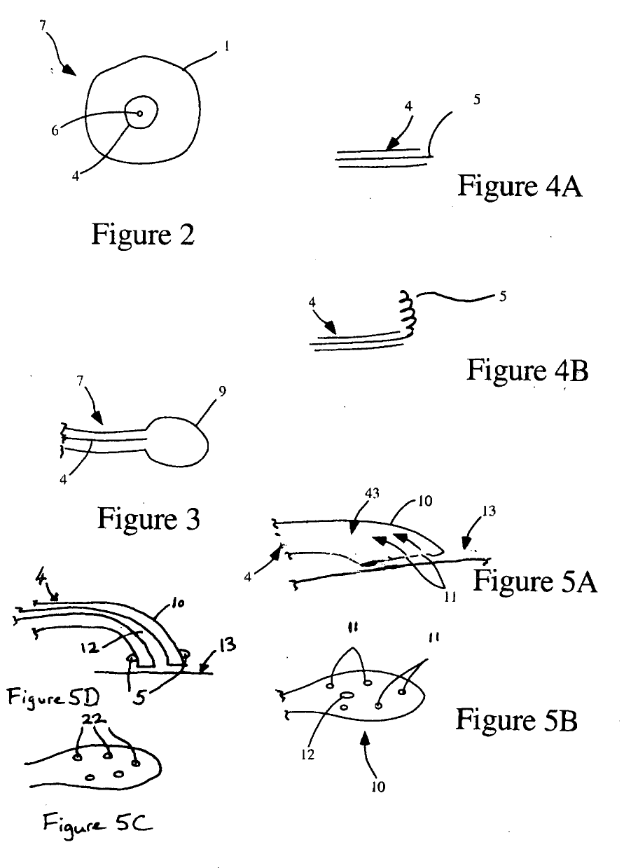

FIG. 2 is a schematic transverse cross-sectional representation of the proximal end of the catheter system.

FIG. 3 shows an alternative embodiment of the distal end of the catheter system showing a balloon (9) inflated to guide the catheter to the apex of the right ventricle.

FIG. 4 shows an alternative embodiment of a combined electrode (5) and attaching means on the distal end of the catheter system showing the tip of the intrapericardial electrical catheter (4) comprising a memory-metal alloy retracted within the lumen of the catheter (FIG. 4A) and when deployed by advancement from out of the lumen of the catheter takes a substantially coiled shape whereby, the coil is screwed from the lead into the myocardium at a perpendicular angle as opposed to parallel out the distal end so that it can attach to the myocardium/epicardium (FIG. 4B).

FIG. 5 shows an alternative embodiment of the distal end of the catheter system showing the tip of the intrapericardial electrical cathetercomprising a suction device (10) having airway apertures (11) and a lumen (12); the tip of the intrapericardial electrical catheteris shaped and adapted for manipulating the intrapericardial electrical catheter against the wall of the mammalian organ (13). FIG. 5A illustrates a longitudinal cross-sectional view; FIG. 5B shows a part of the distal end showing the face of the tip of the intrapericardial electrical catheter that contacts the wall of the mammalian organ illustrating exemplary positions of the apertures and the lumen. FIG. 5C illustrates a part of the distal portion having magnetized material on the face of the tip of the intrapericardial electrical catheter that contacts the wall of the mammalian organ illustrating exemplary positions of the magnetized material. Examples of such materials are magnetized metals or metal alloys. FIG. 5D illustrates an exemplary suction device at the distal end of the intrapericardial electrical catheter.

FIG. 6 shows a detail of the catheter system showing successive stages of a method of using the catheter system. FIG. 6A shows a first tube with a distal attaching mechanism against the inner wall of the right ventricle. FIG. 6A shows the coil attaching and securing the distal ends of the first catheter and it positioned against the wall of the right ventricle. FIG. 6C shows a second tube or perforator perforating the myocardium. This second tube is removed and FIG. 6C shows placement of the intrapericardial electrical catheter. Alternatively, the intrapericardial electrical catheter can be so developed that is can both puncture the myocardium and advance through the myocardium into the pericardial space eliminating the need for a separate perforator. FIG. 6D shows the intrapericardial electrical catheter advanced between the pericardium and the myocardium to the target site upon the surface of the myocardium.

FIG. 7 illustrates a detail of an alternative embodiment of the invention. The second tube further comprises a second electrode (30) affixed near the distal end of the first tube.

FIG. 8 through FIG. 18 show different embodiments of the catheter system of the invention.

FIG. 8 shows a cross-sectional illustration of the mammalian heart showing the catheter (7) positioned within the inferior vena cava (14), the right atrium (15), the right ventricle (16), through a puncture in the myocardial wall (17) of the right ventricle, and within the pericardial space (18). The electrode (5) is positioned for optimal pacing against the left ventricle (19); the wall of the pericardium is also shown (20).

FIG. 9 shows a cross-sectional illustration of the mammalian heart showing the catheter positioned within the superior vena cava (21), the right atrium (15), through a puncture in the myocardial wall (17) of the right atrium, and within the pericardial space (18).

FIG. 10 shows a cross-sectional illustration of the mammalian heart showing an alternative embodiment of the invention. The catheter is positioned within the superior vena cava (21), the right atrium (15), and the right ventricle. The distal end of the intrapericardial electrical catheter comprises a magnet (22) that is guided into position using an electromagnet (23) affixed to a deployment device (24), the deployment device having been inserted into the left ventricle through the femoral artery and aorta. The catheter is placed in the pericardial space as disclosed herein and the electromagnet is used to attract the magnet on the tip of the intrapericardial electrical catheter to position it in a site upon the surface of the heart.

FIG. 11 shows a cross-sectional illustration of the mammalian heart showing the catheter positioned within the superior vena cava (21), through a puncture in the arterial wall (25) of the superior vena cava, and within the pericardial space (18).

FIG. 12A shows a cross-sectional illustration of the mammalian heart showing an alternative embodiment of the invention: a right atrial pacing lead (26) having a an electrode (29) is placed in the right atrium, a right ventricle pacing lead (27) having an electrode (30) is placed in the right ventricle, and a left ventricle pacer (44) is placed in the left ventricle having been inserted using a catheter (28) into the left ventricle through the femoral artery and aorta. The catheter (28) is then removed leaving only the pacer (44). The pacer can be a leadless pacemaker, leadless electrical stimulus, a remotely controlled pacer or selective cells with the ability to pace, or the like. The right atrial pacing lead and right ventricle pacing lead are shown controlled by the pacemaker (PM; 31). The LV pacer interacts remotely or leadlessly with the generator. FIG. 12B shows a detail of the left ventricle pacer (44) showing an exemplary threaded or coiled attaching or securing means (45).

FIG. 13 shows a cross-sectional illustration showing another embodiment of the invention, a single pacing lead having an electrode (5) at the distal end positioned against the left ventricle and a second electrode (30) that in use is positioned at the apex of the right ventricle the single pacing lead having been inserted from the right ventricle to the left ventricle thought the intraventricular septum (50).

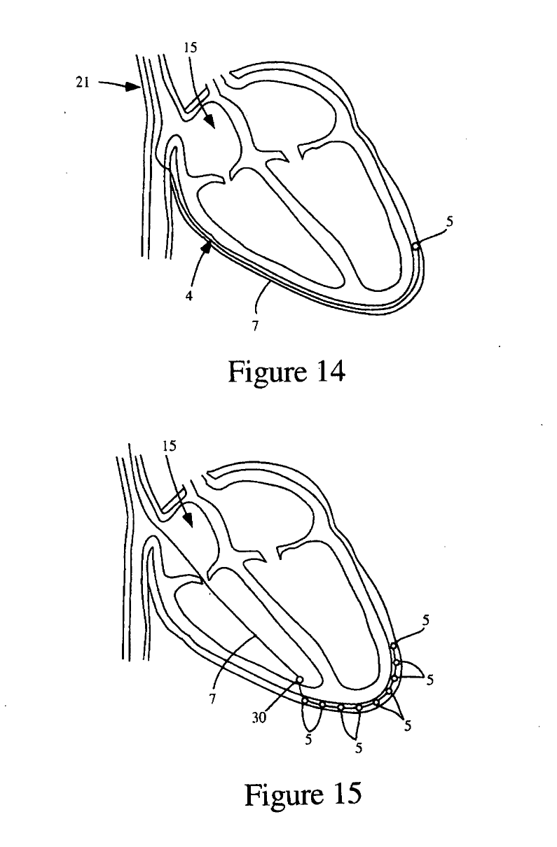

FIG. 14 shows a cross-sectional illustration of the mammalian heart showing the catheter positioned within the superior vena cava (21) and the inferior vena cava, through a puncture in the wall of the inferior vena cava, and positioned within the pericardial space (18).

FIG. 15 shows a cross-sectional illustration showing a preferred embodiment of the invention, a single pacing lead having a plurality of electrodes (5) at the distal end positioned against the left ventricle and a second electrode (30) that in use is positioned at the apex of the right ventricle.

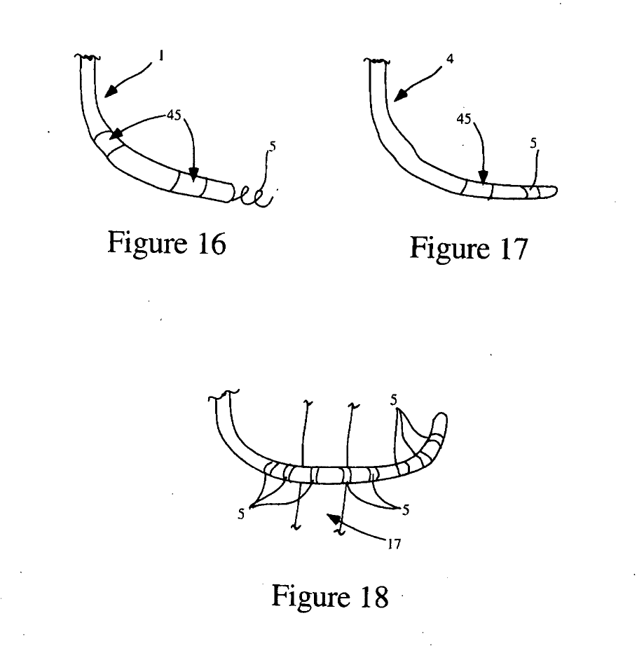

FIG. 16 shows a detail of the distal end of the catheter system of one embodiment of the invention, illustrating the combined electrode (5) and attaching means (coil) and two defibrillation electrodes or coils (45) here positioned on the first tube (1).

FIG. 17 shows a detail of the distal end of the catheter system of one embodiment of the invention, illustrating the electrode (5) and a defibrillation electrode (45) here positioned on the intrapericardial electrical catheter (4).

FIG. 18 shows a detail of the distal end of the catheter system of one embodiment of the invention, illustrating a plurality of electrodes (5) here positioned on the intrapericardial electrical catheter (4) and inserted in a puncture through the myocardium (17).

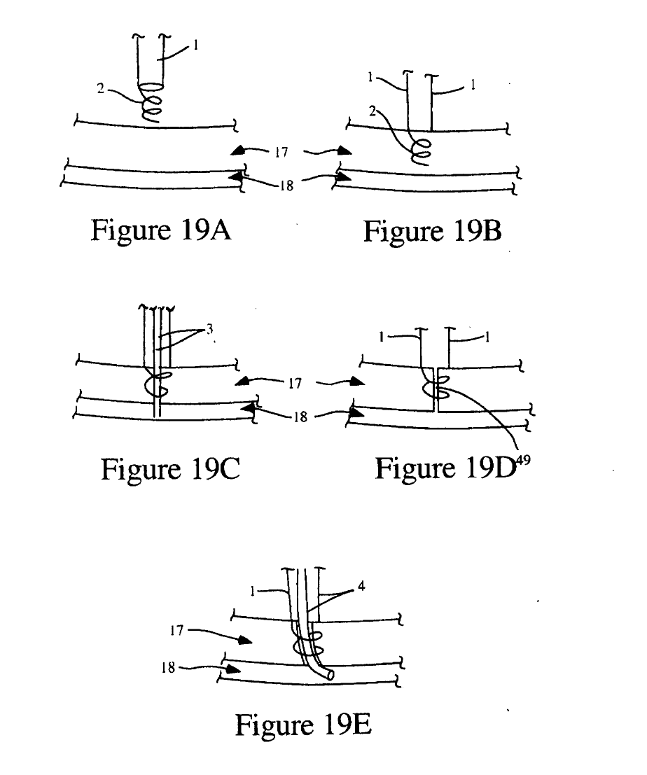

FIGS. 19 through 21 illustrate several embodiments of the invention and how to use them.

FIG. 19 illustrates one embodiment of the invention. FIG. 19A illustrates the first tube (1) comprising attaching means (2) advanced through the thoracic vasculature and in the proximity to the wall of the myocardium (18). The catheter system is advanced towards the wall of the myocardium and concomitantly rotated and the attaching means becomes engaged and attaches to the myocardium (FIG. 19B). A second tube is advanced through the first lumen and punctures and penetrates the myocardium, emerging from the epicedium into the pericardial space (18) (FIG. 19C). The second tube is withdrawn, having created a breach or perforation (49) through the myocardial wall (FIG. 19D). The intrapericardial electrical catheter is advanced through the first lumen, the myocardial perforation and emerges into the pericardial space. Due to the material composition of the intrapericardial electrical catheter, as it is advanced through the myocardial wall it progresses along the curvature of the heart. Alternatively, in another embodiment, flexation of the distal end portion of the intrapericardial electrical catheter allows the intrapericardial electrical catheter to alter the trajectory and be directed parallel to the surface e of the heart. (FIG. 19E).

FIG. 20 illustrates another embodiment of the invention. FIG. 20A illustrates the first tube (1) comprising attaching means (2) advanced through the thoracic vasculature and in the proximity to the wall of the myocardium (18). The catheter system is advanced towards the wall of the myocardium and concomitantly rotated and the attaching means becomes engaged and attaches to the myocardium (FIG. 20B). A second tube is advanced through the first lumen and punctures and penetrates the myocardium, emerging from the epicedium into the pericardial space (18) (FIG. 20C). A 0.014 guidewire (6) is advanced through the third lumen and emerges into the pericardial space (FIG. 20D). The second tube is withdrawn (FIG. 20E) and the intrapericardial electrical catheter is advanced through the first lumen using the guidewire as a rail. The intrapericardial electrical catheter is then advanced over the surface of the heart (FIG. 20F). In this embodiment, the guide-wire may be changed from a straight or slightly curved shape having a curve of between about 0 and 20 degrees to a curved structure having a shape with a curve of between 70 and 90 degrees.

FIG. 21 illustrates another embodiment of the invention. FIG. 21A illustrates the first tube having been attached and anchored in the wall of the myocardium. A 0.035″ guidewire (6) is advanced through the first lumen and upon egress from the myocardium, is contorted by the anatomy of the pericardial space (18) and flexes such that the advancing guidewire remains against and parallel to the epicardium A sheath (48) comprising a substantially flexible material is advanced over the 0.035″ guidewire using the guidewire as a rail. (See FIG. 21A.)

The 0.035″ guidewire is withdrawn, leaving the sheath (48) in position parallel to the surface of the heart (FIG. 21B). A 0.014″ guidewire (6) is advanced through the lumen of the sheath and is advanced towards a site on the surface of the heart (FIG. 21C). The sheath is withdrawn (FIG. 21D) and the intrapericardial electrical catheter (4) is advanced over the 0.014″ guidewire using the guidewire as a rail (FIG. 21E). The intrapericardial electrical catheter is then advanced through the pericardial space to a site on the surface of the heart (FIG. 21F).

FIG. 22 illustrates a detail of a cross-sectional view of a device of the invention placed in the lumen (34) of a blood vessel, the blood vessel being adjacent to the pericardial space. The catheter comprises two balloons (33) fixedly attached near the distal end of the catheter system. Following inflation of the balloons, the catheter is reversibly secured within the blood vessel, and the catheter system and intrapericardial electrical catheter can be deployed within the pericardial space.

FIG. 23 shows a detail of an alternative embodiment of the device placed in a blood vessel. The catheter system comprises an expandable sheet (35) (FIG. 23A) that upon expansion of the sheet is reversible secured in the lumen of the blood vessel (FIG. 23B).

FIG. 24 shows a detail of another embodiment of the device placed in a blood vessel. The catheter system comprises an expandable sealing ring (37) that impinges upon the inner wall of the blood vessel and thereby causing a needle (38) to puncture the wall and allow access to the pericardial space. In one embodiment, needle (38) comprises a dilating mechanism at the distal region. Examples of such dilating mechanisms include, but are not limited to dilating balloons, substantially tapered regions, or the like.

FIG. 25 shows a detail of another embodiment of the catheter device. The catheter device comprises a valve (39) that seals the puncture in the myocardium or blood vessel wall through which the intrapericardial electrical catheter and/or second catheter passes. thereby preventing tamponade.

FIG. 26 shows a detail of a cross section of another embodiment of the catheter device comprising a vacuum steering platform (40) being deployed in use. As shown in FIG. 26A, air is drawn up through a lumen of the catheter system (7) through apertures (11) in the vacuum platform (40). In an alternative embodiment, the vacuum platform can comprise an inflatable balloon (47) that, when inflated during use by pumping air or the like through a lumen (46) into the vacuum plate, exerts a force upon the catheter system (7) causing the catheter system to contort and alter its position relative to the vacuum plate and thereby steers the catheter in a desired direction (Open arrows representing direction of air movement). The steering platforms are of use in individuals lacking a pericardium, for example, individuals having had a coronary bypass surgery performed.

FIG. 27 illustrates a detail of another embodiment of the invention. The second tube comprises a sleeve (41) the sleeve comprising barbs (42) that anchor the catheter device in the wall of the blood vessel or the myocardium. The intrapericardial electrical catheter egresses asymmetrically from the lumen of the second tube through an aperture that is on a side of the distal end of the second tube. The second tube is partially or completely rotated circumpherentialy and independent of the sleeve and the direction of the aperture in the second tube steers the intrapericardial electrical catheter in a desired direction.

FIG. 28 illustrates another embodiment of the invention. FIG. 28A illustrates the first tube (1) directed to (2) in the wall of the myocardium, but not attached. The intrapericardial electrical catheter (4) is illustrated having been advanced through the first lumen, having punctured and penetrated the myocardium (17), and the distal end is within the pericardial space (18). The intrapericardial electrical catheter is then advanced through the pericardial space and against the heart wall (epicardium) in position to pace the heart. (See FIG. 28C.) The first tube is withdrawn from the heart (FIG. 28B) and the RV lead (27) comprising an RV electrode (30) is advanced over the intrapericardial electrical catheter using the intrapericardial electrical catheter as a rail (FIG. 28C). FIG. 29 shows an alternative embodiment of a combined electrode (5) and attaching means on the distal end of the catheter system showing the tip of the intrapericardial electrical catheter (4) comprising a memory-metal alloy retracted within the lumen of the catheter (FIG. 4A) and when deployed by advancement from out of the lumen of the catheter takes a substantially curved shape into the epicardium. The coil is at baseline withdrawn into the catheter and using a delivery mechanism it is embedded into the epicardium/myocardium where it takes on a new shape (for example, having the appearance and shape of a fish hook to prevent it coming out).

### LIST OF REFERENCE NUMERALS

1. First Tube

2. Second Attaching or Securing Means

3. Second Tube

4. Intrapericardial Electrical Catheter

5. First Electrode

6. Guide-wire

7. Catheter System

8. First Attaching or Securing Means

9. Balloon

10. Vacuum Catheter

11. Aperture

12. Lumen

13. Blood Vessel or Chamber

14. Inferior Vena Cava

15. Right Atrium

16. Right Ventricle

17. Myocardium

18. Pericardial Space

19. Left Ventricle

20. Parietal Pericardium

21. Superior Vena Cava

22. Magnet

23. Electromagnet

24. Deployment Device

25. Cardiac Vein

26. RA Lead

27. RV Lead

28. LV Pacer Catheter

29. RA Electrode

30. RV Electrode

31. Pacemaker

32. Inner Surface of Right Ventricle

33. Balloon

34. Blood Vessel Lumen

35. Expandable Sheet

36. Endothelium of Blood Vessel or Right Atrial Appendage

37. Ring

38. Needle

39. Valve

40. Negative/Positive Pressure Steering Platform

41. Sleeve

42. Barb or Screw Thread

43. Vacuum Lumen

44. LV Pacer

45. Defibrillator

46. Positive Pressure Inlet Lumen

47. Inflatable Buttress

48. Sheath

49. Myocardium Perforation

50. Intraventricular Septum

51. Syringe for Deploying Adhesive Compound

### EXAMPLES

The invention will be more readily understood by reference to the following examples, which are included merely for purposes of illustration of certain aspects and embodiments of the present invention and not as limitations.

### Example I

### Adhesive Compounds

Experiment #1: To Find a Good Adhesive to Attach a Device to the Heart Wall

Several adhesives were tested for their suitability in the present invention. Bond strength was tested by using the adhesives to attach a piece of RNF-100 ⅛ heat shrink tubing to a wet myocardium. The bond strength was checked after 30 minutes. The results are as follows:

TABLE 1

| Type of adhesive | Bond strength after 30 minutes |
|---|---|
| Arctic Silver Thermal Adhesive | Low |
| KRAZY GLUE | Medium |
| IPS Weld-On #16 | Very low |
| GOOP Household Contact Adhesive | Very low |
| LOCTITE 4011 | Good |
| LOCTITE Quick Set Epoxy | Low |

Based on the above results, a cyanoacrylate LOCTITE 4011 was chosen for further experiments.

Experiment #2: To Demonstrate Proof of Concept of Using an Adhesive to Attach a Device to the Epicardium.

In this experiment, a length of RNF-100 ⅛ heat shrink tubing was used as an example of a device. Distal end of the tubing was plugged. A series of perforations were done on the distal region of tubing by a needle. The distal end of the tubing was placed on wet epicardium (13). A cyanoacrylate adhesive (LOCTITE 4011) was injected through the proximal end of the tubing using a syringe (51). The adhesive traveled down the lumen of the tubing and emerged out of the perforations (8). The strength of the bond between the tubing and epicardium was checked after 30 minutes. (See FIG. 30.)

Results: A device could be firmly attached to wet epicardium using an adhesive.

### Example II

### Navigation Methods

Several suitable methods and devices are used to navigate one or more devices of the present invention through the anatomy including but not limited to:

1) Devices comprising a pre-set shape that enables them to navigate through the anatomy easily. For example, one or more guidewires disclosed herein may have a pre-set shape that substantially conforms to the curvature of the outer surface of the heart. Such guidewires can be made of suitable materials such as NITINOL.

2) Devices comprising an electromagnetic navigation element that enables the devices to be navigated by a controllable external magnetic field.

These navigation methods and devices are especially useful in patients with a modified anatomy such a patients who have undergone a cardiac surgery that removed the pericardium.

### Example III

### Animal Experiments

Acute Studies

In order to prove our concept and safety, we tested the device and methodology in pigs. After the pig was anesthetized, access to the heart was obtained via the right internal jugular vein. A 10 French introducer sheath was inserted. An RV (right ventricular) lead enclosed by an inner sheathwas introduced through the introducer sheath and positioned in the right ventricular (RV) apex. Maneuverability to the RV apex was achieved using a flexing mechanism of the RV lead. The position of the RV lead was placed and confirmed under fluoroscopy. The inner sheath was partially withdrawn to fully expose a screw/coil on the RV lead. The RV lead was then secured in place by overhand turning the entire inner sheath and the RV lead. After positioning was achieved, pacing threshold and sensitivity were determined and found to be excellent.

After the RV lead was secured in position, a LV (left ventricular) lead was placed via the RV lead. A puncture needle was placed within the RV lead. A pressure wire was placed within the needle. To help confirm perforation into the pericardial space several parameters are measured. The impedance across the needle was measured; the pressure via the pressure wire was measured; current of injury was monitored on the needle. The needle was slowly advanced through the RV lead so as to puncture the RV myocardial wall. In addition to the above parameters, iodinated contrast was injected through the needle to confirm position in the pericardial space.

Once the needle was confirmed to be in the pericardial space, a 0.014″ guidewire was advanced to a location on the LV wall. The needle was withdrawn and the LV lead was placed over the wire to the optimal position. In some cases, the needle was withdrawn after perforation, and a 0.035″ guidewire was advanced through the puncture site to the optimal location. The inner sheath was then placed over the wire to the target position and the 0.035″ guidewire withdrawn. The LV lead was then placed through the sheath to the target position.

In some cases, to save a step, the pressure wire could be also used as the 0.014″ guidewire and directly advanced to position after perforation was confirmed.

In all cases, once the lead was in optimal position pacing thresholds and sensing are determined. Air and fluid was then withdrawn from the pericardial space via the LV lead and pacing thresholds- and sensing are redetermined. In general, they were improved given the better apposition between the lead and the epicardium. Glue was then exuded via the lead to attach it to position. After glue was set, pacing threshold and sensing were redetermined.

In the case of two acute pigs, there was no adverse outcome and no pericardial effusion or tamponade. Pacing thresholds and sensing were within normal limits.

Results

We took an echocardiogram before and after that demonstrated no pericardial effusion before and after. RV pacing threshold was 0.2 mA for animal #1 (this was initially done through a temporary pacing wire so it is in mA); RV pacing was 0.5V @ 0.5 ms for animal #3. RV sensing was 20 mV in animal #1 and 5.9-7.5 mV in animal #3. In animal #3 impedence dropped from 2.7 ohms to 1.8 ohms at the time of puncture into the pericardial space. In animal #1 LV pacing threshold before suction was 5-7 mA. After suctioning to better appose the lead against the tissue the threshold was 0.5 mA and sensing was 5-10 mV. In animal #3 LV pacing threshold was initially 2.4V @ 0.5 ms and at points there was diaphragmatic stimulation. With easily maneuvering the lead (forward or back) the final numbers were threshold 0.8V @ 0.5 ms and sensing was 6-8 mV with no diaphragmatic stimulation. Total time from start (insert RV lead into vein) to finish (LV lead placed and attached) in animal #3 was 1 hour.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

We claim:

1. A catheter system for positioning an intrapericardial electrical catheter against a site upon the surface of a mammalian heart, the mammalian heart having an inner surface and an outer surface defining a wall, the catheter system comprising:
a first tube, the first tube having a proximal end and a distal end, and defining a first lumen within the longitudinal axis of the first tube,
an attaching means having a proximal end and a distal end, wherein the proximal end of the attaching means is affixed to the first tube at or near the distal end of the first tube and the distal end of the attaching means is adapted for attaching the first tube to the wall of the mammalian heart,
a second tube, the second tube having a distal end adapted to puncture the wall of the mammalian organ and having a proximal end, wherein the second tube is adapted for placement within the first lumen, and
an intrapericardial electrical catheter having a distal end and a proximal end, defining a second lumen within the longitudinal axis of the intrapericardial electrical catheter, wherein the intrapericardial electrical catheter is adapted for insertion through the first lumen, and wherein the distal end of the intrapericardial electrical catheter is shaped and adapted for placement against a site upon the surface of the mammalian heart.

2. The catheter system of claim 1, wherein the attaching means is selected from the group consisting of stylet, a hook, a clip, a staple, an adhesive, a coil, a barb, a serrated blade or knife, a threaded screw, and a vacuum device.

3. The catheter system of claim 1, wherein the distal end of the intrapericardial electrical catheter further comprises attaching means selected from the group consisting of stylet, a hook, a clip, a staple, an adhesive, a coil, a barb, a serrated blade or knife, a threaded screw, and a vacuum device.

4. The catheter system of claim 1, wherein the first tube further comprises at least one electrode.

5. The catheter system of claim 4 wherein the at least one electrode is selected from the group consisting of a pacing electrode and a defibrillating electrode.

6. The catheter system of claim 1, the first tube further comprising a flexing element.

7. The catheter system of claim 1, wherein the first catheter further comprises a gasket ring, wherein the gasket ring is affixed near the distal end of the first catheter and wherein the gasket ring has a diameter more than that of the first catheter.

8. The catheter system of claim 1, the proximal end and the distal end of the second tube defining a third lumen.

9. The catheter system of claim 1, further comprising a guide-wire.

10. The catheter system of claim 1 wherein the intrapericardial electrical catheter is concentrically disposed within the first lumen and comprises at least one electrode, and the electrode is in electrical conductivity to a power source at or near the proximal end of the intrapericardial electrical catheter.

11. The catheter system of claim 10 wherein the first catheter further comprises at least one second electrode, the second electrode being disposed at or near the distal end of the second catheter and is in electrical conductivity to a power source at or near the proximal end of the first tube and wherein the wall of the mammalian organ conducts a current when a potential difference is created between the first electrode and the second electrode using the power source.

12. The catheter system of claim 11, wherein the potential difference is not more than about 2.5 V at 0.5 ms.

13. The catheter system of claim 1, wherein the distal end of the intrapericardial electrical catheter further comprises a marker, the marker selected from the group consisting of a dye, a radio-opaque material, a magnet, and an ion source.

14. The catheter system of claim 3 wherein the attaching means are adapted for securing the intrapericardial electrical catheter to a heart wall

15. The catheter system of claim 1 wherein the catheter system further optionally comprises a catheter selected from the group consisting of an ablation catheter, a cryogenic catheter, a drug-delivery catheter, and a cell-delivering catheter.

16. The catheter system of claim 1 wherein the intrapericardial electrical catheter further comprising at least one electrode, wherein the electrode is selected from the group consisting of a pacing electrode and a defibrillating electrode.

17. A method of using the catheter system of claim 1 to treat an individual having a cardiac pathology, the method comprising the steps of:
(i) providing an individual having a cardiac pathology,
(ii) providing the catheter system of claim 1,
(iii) advancing the first tube through the thoracic circulatory system, the thoracic circulatory system selected from the group consisting of the inferior vena cava, the superior vena cava, the carotid sinus, the right atrial appendage of the heart, the right atrium, the left atrium, the right ventricle, and the left ventricle of the individual,
(iv) advancing the distal end of the first tube against the inner wall of the right ventricle,
(v) rotating and advancing the first tube and optionally engaging the coil with the tissue of the myocardium whereby the coil punctures and attaches to the myocardium and anchoring the system in the myocardium thereby,
(vi) advancing the second tube (through the first lumen and puncturing the myocardium,
(vii) advancing the second tube through the punctured myocardium and penetrating the myocardium thereby,
(viii) advancing the second tube through the myocardium and penetrating the outer surface of the myocardium,
(ix) advancing the intrapericardial electrical catheter through the first lumen,
(x) advancing the intrapericardial electrical catheter across the outer surface of the myocardium to a site on the outer surface of the myocardium, and
(xi) operating the intrapericardial electrical catheter, thereby treating the individual having a cardiac pathology and that results in the patient having an improved heart function.

18. The method of claim 17 further comprising a step of flexing the first tube upon egress from the myocardium.

19. The method of claim 17 further comprising a step of counter-rotating and withdrawing the first tube and disengaging the coil from the myocardium.

20. The method of claim 17 further comprising a step of advancing at least one guidewire through the second tube.

21. The method of claim 20 wherein the guidewire is selected from the group consisting of a 0.035″ guidewire and a 0.014″ guidewire and the guidewire advanced through the second tube is a 0.035″ guidewire, further comprising the steps of (xii) advancing a sheath over the 0.035″ guidewire, the sheath having a lumen, (xiii) removing the 0.035″ guidewire, (xiv) advancing a 0.014″ guidewire through the lumen of the sheath, and (xv) removing the sheath.

22. A system for diagnosing, preventing or treating a cardiac pathology by perforating through a cardiac tissue and placing a medical device or fluid in a heart chamber comprising:
a) an elongated, tubular delivery device having a delivery device lumen extending from a delivery device proximal region to a delivery device distal region and a distal fixation mechanism at the delivery device distal region adapted to be advanced into the heart and fixed to a selected site on the heart wall;
b) an elongated perforating device having an perforating device distal tip adapted to be advanced through the delivery device lumen and perforate through the cardiac tissue into the heart chamber to form a perforation; and
c) an elongate medical device for diagnosing, preventing or treating a cardiac pathology to be advanced through the perforation into the heart chamber.

23. The system of claim 22 wherein the cardiac pathology is an electrophysiological pathology

24. A system for diagnosing, preventing or treating a cardiac pathology by perforating through a thoracic vascular tissue and placing a medical device or fluid in a heart chamber comprising:
a) an elongated, tubular delivery device having a delivery device lumen extending from a delivery device proximal region to a delivery device distal region;
b) an elongated perforating device having an perforating device distal tip adapted to be advanced through the delivery device lumen and perforate through the thoracic vascular tissue into the heart chamber to form a perforation; and
c) an elongate medical device for diagnosing, preventing or treating a cardiac pathology to be advanced through the perforation into the heart chamber.

25. The system of claim 24 wherein the cardiac pathology is an electrophysiological pathology.

26. A system for delivering electrical energy to multiple cardiac locations by perforating through a cardiac tissue and placing a medical device comprising:
a) an elongated, tubular delivery device having a delivery device lumen extending from a delivery device proximal region to a delivery device distal region;
b) an elongated perforating device having an perforating device distal tip adapted to be advanced through the delivery device lumen and perforate through the cardiac tissue into the pericardial space to form a perforation; and
c) an elongate medical device for delivering electrical energy to multiple cardiac locations to be advanced through the perforation into the pericardial space, wherein the elongate medical device comprises two or more distal electrodes for delivering electrical energy and two or more proximal electrodes for delivering electrical energy.
